(19) United States
(12) Patent Application Publication
(10) Pub. No.: US 2022/0154163 A1
(43) Pub. Date: May 19, 2022

(54) HIGH-PRECISION BASE EDITORS

(71) Applicant: MAX-PLANCK-GESELLSCHAFT ZUR FÖRDERUNG DER WISSENSCHAFTEN E.V., München (DE)

(72) Inventors: Ralph BOCK, Schwielowsee (DE); Daniel KARCHER, Potsdam (DE); Junjie TAN, Potsdam (DE)

(21) Appl. No.: 17/438,543

(22) PCT Filed: Jan. 30, 2020
(86) PCT No.: PCT/EP2020/052368

§ 371 Date: Sep. 13, 2021

(30) Foreign Application Priority Data
Mar. 13, 2019 (EP) PCT/EP2019/056335

(51) Int. Cl. C12N 9/78; C12N 9/22; C12N 15/11; C12N 15/86; C12N 15/90; A61K 31/7088; A61K 38/46; A61K 38/50

(52) U.S. Cl. CPC C12N 9/78; C12N 9/22; C12N 15/11; C12Y 305/04; C12N 15/86; C12N 15/907; C12N 2710/16143; A61K 38/465; A61K 38/50; C12N 2310/20; C12N 2800/80; C12Y 305/04005; A61K 31/7088

(57) ABSTRACT

The present invention relates to a base editing compound comprising or consisting of (a) a Cas protein, and, covalently connected therewith; (b) a nucleobase-modifying enzyme, wherein the covalent connection of (a) and (b) is (i) direct; (ii) provided by a peptide comprising at least one Pro residue, said peptide having a length between 1 and 20 preferably between 1 and 15 amino acids; or (iii) provided by a non-peptidic linker, said non-peptidic linker being a small organic molecule comprising one or more double bonds, one or more triple bonds, and/or one or more aromatic rings.

The present invention relates to a base editing compound comprising or consisting of (a) a Cas protein, and, covalently connected therewith; (b) a nucleobase-modifying enzyme, wherein the covalent connection of (a) and (b) is (i) direct; (ii) provided by a peptide comprising at least one Pro residue, said peptide having a length between 1 and 20, preferably between 1 and 15 amino acids; or (iii) provided by a non-peptidic linker, said non-peptidic linker being a small organic molecule comprising one or more double bonds, one or more triple bonds, and/or one or more aromatic rings.

In this specification, a number of documents including patent applications and manufacturer's manuals are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

CRISPR-Cas systems represent an adaptive immune system in bacteria that promotes antiviral defense (Jinek, M. et al. A programmable dual-RNA-guided DNA endonuclease in adaptive bacterial immunity. Science 337, 816-821 (2012); van der Oost, J., Westra, E. R., Jackson, R. N. & Wiedenheft, B. Unravelling the structural and mechanistic basis of CRISPR-Cas systems. Nat. Rev. Microbiol. 12, 479-492 (2014)). Several such systems, especially the one based on the Cas9 enzyme from Streptococcus pyogenes (SpCas9), have been successfully repurposed for genome editing in a wide range of organisms (Mali, P. et al. RNA-guided human genome engineering via Cas9. Science 339, 823-826 (2013); Jiang,

W., Bikard, D., Cox, D., Zhang, F. & Marraffini, L. A. RNA-guided editing of bacterial genomes using CRISPR-Cas systems. Nat. Biotechnol. 31, 233-239 (2013); Nekrasov, V., Staskawicz, B., Weigel, D., Jones, J. D. G. & Kamoun, S. Targeted mutagenesis in the model plant Nicotiana benthamiana using Cas9 RNAguided endonuclease. Nat. Biotechnol. 31, 691-693 (2013); Doudna, J. A. & Charpentier, E. The new frontier of genome engineering with CRISPR-Cas9. Science 346, 1258096 (2014); Wright, A. V., Nunez, J. K. & Doudna, J. A. Biology and applications of CRISPR systems: harnessing nature's toolbox for genome engineering. Cell 164, 29-44 (2016); Komor, A. C., Badran, A. H. & Liu, D. R. CRISPR-based technologies for the manipulation of eukaryotic genomes. Cell 168, 20-36 (2017)). Cas9 is an endonuclease with two nuclease domains, referred to as HNH and RuvC, each cleaving one strand of the target DNA (Jinek, M. et al. Structures of Cas9 endonucleases reveal RNA-mediated conformational activation. Science 343, 1247997 (2014); Nishimasu, H. et al. Crystal structure of Cas9 in complex with guide RNA and target DNA. Cell 156, 935-949 (2014)). The HNH nuclease cleaves the target strand, and the RuvC nuclease cleaves the non-target strand. Upon repair of the double-strand break, deletions (or insertions) can occur that inactivate the target gene (Cong, L. et al. Multiplex genome engineering using CRISPR/Cas systems. Science 339, 819-823 (2013)).

Although this method provides a highly efficient tool in functional genomics and is also suitable to reach a limited number of breeding goals by knocking out genes for unwanted traits in crops (Shan, Q. et al. Targeted genome modification of crop plants using a CRISPR-Cas system. Nat. Biotechnol. 31, 686-688 (2013); Zhu, C. et al. Characteristics of genome editing mutations in cereal crops. Trends Plant Sci. 22, 38-52 (2016)), more precise DNA editing tools are needed for all applications requiring introduction of specific base changes into target genes, such as precision breeding and gene therapy. Most hereditary diseases in humans involve single point mutations, the correction of which will require extraordinary accuracy of site-specific editing, ideally without any off-target effects (Mali, P. et al. CAS9 transcriptional activators for target specificity screening and paired nickases for cooperative genome engineering. Nat. Biotechnol. 31, 833-838 (2013); Hsu, P. D. et al. DNA targeting specificity of RNA-guided Cas9 nucleases. Nat. Biotechnol. 31, 827-832 (2013)).

Recently, base editors have been developed that convert Cas endonucleases into programmable nucleotide deaminases (Komor, A. C., Kim, Y. B., Packer, M. S., Zuris, J. A. & Liu, D. R. Programmable editing of a target base in genomic DNA without doublestranded DNA cleavage. Nature 533, 420-424 (2016); Nishida, K. et al. Targeted nucleotide editing using hybrid prokaryotic and vertebrate adaptive immune systems. Science 353, 1248 (2016) ; Gaudelli, N. M. et al. Programmable base editing of A.T to G.C in genomic DNA without DNA cleavage. Nature 551, 464-471 (2017)), thus facilitating the introduction of C-to-T mutations (by C-to-U deamination) or A-to-G mutations (by A-to-I deamination) without induction of a double-strand break (Rees, H. A. et al. Improving the DNA specificity and applicability of base editing through protein engineering and protein delivery. Nat. Commun. 8, 15790 (2017); Kim, J. -S. Precision genome engineering through adenine and cytosine base editing. Nat. Plants 4, 148-151 (2018)).

As a rule, base editors edit the non-target strand. Komor et al. (loc. cit.) introduce the notion of three generations of base editors. First generation base editors (BE1) make use of a dead Cas9 protein, i.e. a Cas9 protein where both HNH and RuvC endonucleases are inactivated. A typical mutation which inactivates RuvC is D10A. A typical mutation which inactivates HNH is H840A. Fused to such dead Cas9 protein, base editors of the first generation comprise a cytidine deaminase enzyme. The cytidine deaminase enzyme is generally located N-terminally and connected to the dead Cas9 protein via linker.

Subsequently, it has been discovered that endogenous base-excision repair (BER) mechanisms cause reversion of any base editing performed by the cytidine deaminase. In order to control such BER, a third fusion partner has been introduced, namely an inhibitor of base-excision repair such as a uracil DNA glycosylase inhibitor (UGI). Accordingly, such second generation base editors (BE2) are tripartite fusion proteins. As regards the Cas9 protein, use is made of dead Cas9.

In a next step, it has been discovered that eukaryotic mismatch repair (MMR) can be biased towards replacing G (which base paired with C prior to the action of the cytidine deaminase) with A. It turned out that this can be achieved by using a Cas9 nickase instead of dead Cas9, wherein in the Cas9 nickase to be used in base editors, the His at position 840 which is within the HNH sequence is brought back and only the Asp10Ala mutation in the RuvC domain is retained. A Cas9 nickase (nCas9) cleaves only one of the two strands, wherein the specific nCas9^{D10A} only nicks the target strand which is the non-edited strand. Base editors of the third generation (BE3), a preferred starting point for the developments leading to the present invention, accordingly comprise a nickase form of SpCas9 (nSpCas9, to stimulate cellular DNA mismatch repair) fused to a nucleobase deaminase enzyme as well as an inhibitor of base excision repair such as uracil glycosylase inhibitor (UGI).

The current severe limitation in the applicability of base editors lies in their low site selectivity. For example, C-to-T base editors can potentially edit any C that resides in an approximately 4-5 nt (in some systems up to 9 nt) wide window within the protospacer (Komor, A. C., Kim, Y. B., Packer, M. S., Zuris, J. A. & Liu, D. R. Programmable editing of a target base in genomic DNA without doublestranded DNA cleavage. Nature 533, 420-424 (2016); Nishida, K. et al. Targeted nucleotide editing using hybrid prokaryotic and vertebrate adaptive immune systems. Science 353, 1248 (2016); Zong, Y. et al. Precise base editing in rice, wheat and maize with a Cas9-cytidine deaminase fusion. Nat. Biotechnol. 35, 438-440 (2017)). However, some human disease-associated alleles such as the Alzheimer's disease-associated gene APOE4 and the β-thalassemia locus HBB have multiple Cs around the targeted C within the activity window, and the editing of additional Cs can potentially cause deleterious effects ((Komor, A. C., Kim, Y. B., Packer, M. S., Zuris, J. A. & Liu, D. R. Programmable editing of a target base in genomic DNA without double-stranded DNA cleavage. Nature 533, 420-424 (2016); Liang, P. et al. Correction of β-thalassemia mutant by base editor in human embryos. Protein Cell 8, 811-822 (2017)).

Another limitation of the known base editors is the fact that the Cas system, for proper recognition of a target site, requires presence of a so-called “protospacer adjacent motif (PAM)”. In the case of Cas9, the canonical PAM is 5′-NGG-3′, wherein N may be any base. This limits the applicability of Cas9-based base editors to target sites comprising such PAM. The majority of the described base editors are Cas9-based base editors.

In view of the shortcomings of the prior art, the technical problem underlying the present invention can be seen as a provision of improved means and methods for the editing of nucleobases.

This technical problem has been solved by the subject-matter of the claims.

Accordingly, in a first aspect, the present invention relates to a base editing compound comprising or consisting of (a) a Cas protein, and, covalently connected therewith; (b) a nucleobase-modifying enzyme, wherein the covalent connection of (a) and (b) is (i) direct; (ii) provided by a peptide comprising at least one Pro residue, said peptide having a length between 1 and 20, preferably between 1 and 15 amino acids; or (iii) provided by a non-peptidic linker, said non-peptidic linker being a small organic molecule comprising one or more double bonds, one or more triple bonds, and/or one or more aromatic rings.

The term “base editing” refers to the capability of converting a nucleobase into another nucleobase. Nucleobases in accordance with the disclosure are adenine, guanine, inosine, cytosine, thymidine and uracil. Preferably, a purine is converted into another purine, and a pyrimidine into another pyrimidine. Examples of the former include the conversion of adenine to inosine. Inosine may be subsequently converted into guanine, e.g., in the course of DNA replication. An example of the latter is the conversion of cytosine to thymine or uracil. Also envisaged is the conversion of guanine to adenine and of thymidine to cytidine.

The term “base editing” also extends to the capability of methylating or demethylating nucleobases. For example, side-specific cytosine methylation or demethylation is a means of introducing epigenetic changes, also referred to as “epigenome-editing”. Suitable enzymes are disclosed further below.

Base editing compounds in accordance with the present invention are or are predominantly polypeptidic in nature. More specifically, base editing compounds in accordance with the invention comprise at least two components (a) and (b), a targeting component (a) and an editing component (b). The targeting component makes use of the sequence-dependent recognition of target sites by the CRISPR/Cas system. A targeting component making use of the sequence-dependent recognition of target sites by the CRISPR/Cas system is also referred to as “Cas protein” herein. As such, it is understood that a “Cas protein” in accordance with the invention, when provided with a guide sequence as discussed further below, is capable of associating with a region on a target nucleic acid which is complementary or substantially complementary to said guide nucleic acid. Preferably, it is a Cas nickase (nCas) or a (catalytically) dead Cas (dCas). To the extent the targeting component is a Cas nickase, it preferably exhibits enzymatic activity. Preferably, of the two cleavage activities of a native Cas protein, cleavage activity acting on the non-edited strand is obtained. Not necessarily, but preferably, base editors including base editors of the present invention edit the non-target strand, the term “target strand” in this context referring to the DNA strand which is complementary to the guide RNA of the Cas complex. Accordingly, in the preferred embodiment where Cas is Cas9, and furthermore use is made of nCas9, said nCas9 is nCas9^{D10A}. In other words, the RuvC domain is catalytically inactive. The other nuclease domain (HNH) is active, i.e., there is a His at position 840.

Using the nomenclature introduced further above, a base editor using a Cas nickase is a base editor of the third generation. Base editors of first and second generation use a dead Cas protein.

Also preferred is the use of Cas9 variants, more specifically variants of nCas9. These variants include VQR-Cas9, VRER-Cas9, xCas9, and SpCas9-NG. These Cas9 variants are described in more detail in the Examples enclosed herewith and the references cited there.

Base editors of the third generation (BE3) are preferred, because they contain an inhibitor of base excision repair and make use of a Cas nickase. Corresponding constructs of the present invention contain the abbreviation “BE3”.

The PAM sequences recognized by these four nCas9 variants are as follows: NGA in case of VQR-Cas9; NGCG in case of VRER-Cas9; NG, GAA and GAT in case of xCas9; and NG in case of SpCas9-NG.

The other component (b) is capable of catalyzing at least one of the above described nucleobase conversions. Preferred classes of component (b) are cytidine deaminases and adenosine deaminases. Cytidine deaminases are particularly preferred.

Owing to the fact that the two components are covalently connected to each other, the nucleobase-modifying enzyme will exert its function only in the region of the target nucleic acid recognized by the targeting component.

The target may be DNA or RNA. Preferably, the target nucleic acid is DNA. The term “target” or “target nucleic acid” refers to the nucleic acid to be edited by the base editing compound of the invention. The term “target site” refers to a sub-sequence within the target where the nucleobase conversion is to occur. The targeting of the target site within the target nucleic acid is effected by the known sequence-dependent recognition mechanism of the CRISPR/Cas system. In particular, and this is subject of embodiments disclosed further below, the base editing compound of the invention is to be used in conjunction with a guide nucleic acid, preferably a guide ribonucleic acid.

As noted above, the term “target” is also used herein in the context of a “target strand”. The target strand is that strand of the double-stranded DNA which base pairs with the guide RNA.

In relation to both the Cas nickase and the nucleobase-modifying enzyme, it is preferred that they consist of those residues which are required for their respective function. Flanking residues at either the N-terminus or the C-terminus or both, to the extent they do not significantly contribute to function, are preferably absent. As will become more apparent below, the present inventors discovered that in certain instances significant deletions are possible.

On the other hand, the present invention may also make use of Cas nickases and nucleobase-modifying enzymes as they are comprised in base editors of the prior art. Such—not truncated (when using the terminology of this disclosure)—constituents are, for example, the Cas9 nickase as set forth by the amino acid sequence of SEQ ID NO: 1, the APOBEC1 deaminase of SEQ ID NO: 3 and the CDA1 deaminase of SEQ ID NO: 5. APOBEC1 and CDA1 are preferred cytidine deaminases.

The present inventors surprisingly discovered that a fine-tuning of the connection between the two components of the base editing compound is a means of enhancing the precision of base editing. The specific solutions in accordance with the present invention are described by items (i) to (iii) of the first aspect of the invention. Generally speaking, key features of the connection between the two components are (1) limited length, and (2) rigidity.

It is understood that the term “direct” refers to a linkerless connection of components (a) and (b). Preferably, it refers to a main chain peptide bond between the C-terminus of one of the components and the N-terminus of the other component.

The term “editing profile” as used herein refers to the editing properties of a base editor such as the base editing compound in accordance with the invention. The notion of the editing profile includes (i) precision, (ii) location of the edited position relative to the PAM motif, and (iii) efficacy. The main focus of the present invention lies on precision, and furthermore on aspect (ii). To explain further, and as described in the introductory section, base editors of the prior art suffer from deficiencies in that the editor, once it has bound to its target site on the target nucleic acid, performs several nucleobase conversions or single-nucleotide conversions at different positions, albeit in a window which is usually less wide than 15 nucleotides. For many applications, in particular in the field of therapy, this is unacceptable. It turns out that the specific linker design in accordance with the invention is a means of sharpening the editing profile or, in other words, increasing editing precision such that a very limited number of nucleobases at a given site are converted. Preferably, exactly one base is converted. Corresponding evidence can be found in the enclosed Examples.

Having regard to rigidity, the presence of at least one Pro residue in accordance with item (i) is a means of conferring rigidity. To the extent a non-peptidic linker is used, the rigidity conferred by is preferably at least that conferred by a peptidic linker in accordance with item (i). A preferred reference state for defining rigidity of non-peptidic linker is the rigidity of the most preferred peptidic linkers with the sequences PAPAP (SEQ ID NO: 15) and PAPAPAP (SEQ ID NO: 16). Preferred structural implementations of the non-peptidic linker are recited in item (iii).

In a preferred embodiment, (a) said Cas protein is a Cas nickase or dead Cas, said Cas nickase preferably being Cas9 or Cas12 nickase (nCas9; nCas12), and said dead Cas preferably being dead Cas9 or dead Cas12; and/or (b) said nucleobase-modifying enzyme is selected from a deaminase, a nucleoside synthase, a DNA methyl transferase and a DNA demethylase, said deaminase preferably being selected from the APOBEC, CDA1 or Tad/ADAR families, APOBEC3A (abbreviated as “A3A”) being particularly preferred.

To the extent use is made of nCas9, it is also envisaged to make use of that version of nCas9 which is a component of a base editor of the third generation which is referred to as “high-fidelity base editor (HF-BE3)” in Rees et al. (Rees, H. A. et al. Improving the DNA specificity and applicability of base editing through protein engineering and protein delivery. Nat. Commun. 8, 15790 (2017)). These mutations are the following four: N497A, R661A, Q695A and Q926A.

APOBEC is an abbreviation for “apolipoprotein B mRNA editing enzyme, catalytic polypeptide-like”. The term designates a family of cytidine deaminases. The APOBEC family comprises APOBEC1, APOBEC2, APOBEC3A, APOBEC3B, APOBEC3C, APOBEC3D (sometimes are also referred to as APOBEC3E), APOBEC3F, APOBEC3G, APOBEC3H, APOBEC4 and activation-induced (cytidine) deaminase.

The abbreviation CDA1 stands for “cytidine deaminase 1”.

The abbreviation “ADAR” stands for adenosine deaminase acting on RNA”. It is understood that ADAR-containing base editors are capable of editing DNA and/or RNA.

Within the APOBEC family, APOBEC3A and APOBEC1 are preferred.

Within the Tad/ADAR family, TadA and ADAR2 are preferred.

In a further preferred embodiment, said deaminase is truncated at the N- and/or C-terminus, wherein in case of APOBEC deaminases C-terminal truncation is preferred, an APOBEC3A with a C-terminal truncation of 17 amino acids (A3AΔ182) being particularly preferred, and in case of CDA1 deaminases, truncations from residue 188 or residue 198 onwards being preferred. The present inventors found out that deaminases are amenable to truncation. In other words, N- and/or C-terminal residues may be removed without a significant impact on catalytic activity.

In a further preferred embodiment, said compound comprises said peptide and wherein said peptide (i) consists of an amino acid sequence comprising 1 to 10 Pro residues and 1 to 10 small amino acid residues, said small amino acid residues preferably being selected from Ala, Gly, Cys and Ser, said amino acid sequence preferably being the sequence of SEQ ID NO: 130 (XTEN); (ii) has a length of 5, 6 or 7 amino acids; and/or (iii) consists of the sequence Aₘ(PA)ₙPₚ, wherein m and p are independently 0 or 1 and n is 1, 2, 3, 4, 5, 6 or 7, for example of SEQ ID NO: 154 or 162.

While the XTEN linker has been used in a number of proof-of-principle experiments disclosed in more detail further below, it is noted that the present inventors' contribution extends to the design of improved linkers connecting deaminase and Cas protein. Such improved linkers are the proline-rich alternating sequences of item (iii), for example the sequences of SEQ ID NOs: 154 or 162. Also these linkers have been reduced to practice. It is furthermore expected that replacing the XTEN linker with such proline-rich linker in accordance with the invention leads to further improvements.

This preferred embodiment relates to those base editing compounds of the invention which have a peptidic linker connecting components (a) and (b). As a consequence, such base editing compounds of the invention are made of a single polypeptide chain. While this is not a requirement, it is preferred. To explain further, when the agent of the invention is a single polypeptide chain, it may be delivered to the cell in the form of a nucleic acid encoding it. Such nucleic acid is also an aspect of the invention which is disclosed further below.

Small amino acid residues include Ala, Gly, Ser, Cys, Thr and Val, the first four being preferred. Preference is given to a plurality of prolines being interspersed in a sequence of small amino acids. This may give rise to a regular pattern as defined in item (iii) of this preferred embodiment, but does not have to. In other words, while particular preference is given to the sequences of SEQ ID NOs: 15 and 16, also peptidic linker consisting of sequences such as PX₂P(XP)ₖ or PXP₂(XP)ₖ and the like may be used, wherein “X” designates a small amino acid to be chosen from the above specific amino acids independently for each occurrence of X, an k is 0, 1, 2, 3, 4 or 5, preferably 1.

Said amino acid sequence of (i) may consist exclusively of the recited 1 to 10 Pro residues and 1 to 10 small amino acid residues, but does not have to. To give a specific example, the linker sequence designated “XTEN” herein, while comprising two prolines and a number of small amino acids, also comprises other amino acids, and constitutes a linker to be used in conjunction with the present invention.

In a further preferred embodiment, the truncated residues are not essential for catalytic activity of said deaminase.

The two preferred strategies in accordance with the present invention, namely truncation of the nucleobase-modifying enzyme and conferring rigidity to the linker connecting targeting moiety and modifying moiety may be used independently or in combination.

In a preferred embodiment, said base editing compound comprises or further consists of (a) an inhibitor of base excision repair, preferably an uracil DNA glycosylase inhibitor (UGI), more preferably the sequence of SEQ ID NO: 149, wherein said UGI is fused to said Cas protein or said nucleobase-modifying enzyme; and/or (b) a nuclear localization signal (NLS), preferably the sequence of SEQ ID NO: 135; wherein (a) and/or (b) are preferably connected to each other and/or to said Cas protein with a peptidic linker consisting of 1 to 10 amino acids, said linker preferably consisting of the sequence of SEQ ID NO: 132 or 148. Using the art-established nomenclature, an inhibitor of base excision repair such as UGI is a feature of base editors of the second and third generation. These base editors are accordingly (at least) tripartite fusion constructs. Base editors of the first generation (BE1) are generally bipartite fusion constructs because they do not comprise an inhibitor of base excision repair.

Preferred orders of the fusions comprising Cas protein deaminase UGI and NLS are apparent, for example, from the sequences of the most preferred base editors listed in Table 1 further below. Generally speaking, the inhibitor of base excision repair is preferably fused to the C-terminus of the Cas protein, and/or the NLS is preferably fused to the C-terminus of the inhibitor of base excision repair. In either case, short peptidic linkers, for example consisting of 1 to 10 amino acids, may be used.

The phrase “further consists of” is used to describe those embodiments which consist of the constituents recited in the broadest definition of the first aspect of the invention and the further constituents recited in the preferred embodiment at issue. In other words, the closed language “consisting of” is maintained with regard to this preferred embodiment in that said preferred embodiment presents a closed list of all constituents required and allowed to be present in the base editing compound of the invention in accordance with the particular preferred embodiment at issue.

Uracil glycosylase inhibitors are known in the art. A preferred sequence thereof is disclosed further below. In functional terms, uracil glycosylase inhibitors implement the generic notion of inhibitors of base excision repair. They serve to improve the yield or efficacy of base editing in that a higher proportion of the desired editing result is obtained on both complementary strands of the DNA to be edited.

NLS sequences are known in the art. A preferred NLS sequence is disclosed further below.

In line with the use of the term “fused” as herein, it includes both direct fusions with no intervening amino acids or linkers (“linkerless fusion”) as well as those fusions wherein between the two components to be fused, there is a peptidic linker. Preferably said peptidic linker consists of 1 to 10 amino acids, said amino acids preferably being selected from Gly and Ser. Exemplary peptidic linkers connecting UGI and NLS to base editing compounds of the invention are apparent from specific constructs of the invention is discussed further below.

There is no particular order of the components of the base editing compound of the invention, with the exception of the targeting component (preferably a Cas protein) and the editing component (preferably a nucleobase-modifying enzyme) being connected as defined by any one of items (i) to (iii) of the first aspect. In other words, UGI and independently NLS may be upstream or downstream of both the Cas protein and the nucleobase-modifying enzyme.

In a further preferred embodiment, (a) said deaminase is APOBEC3A (A3A; SEQ ID NO: 183), wherein preferably said A3A is truncated at the C-terminus; (b) said deaminase is A3AΔ182 (SEQ ID NO: 205) and is fused to the N-terminus of said Cas protein; (c) said deaminase is APOBEC1, preferably consisting of the sequence of SEQ ID NO: 129, and is fused to the N-terminus of said Cas protein, preferably of said Cas nickase; or (d) said deaminase is CDA1, preferably consisting of the sequence of SEQ ID NO: 137; and is fused to the N-terminus or the C-terminus, preferably to the N-terminus of said Cas protein, preferably of said Cas nickase, wherein preferably the C-terminus of said CDA1 is truncated, preferably either from position 198 onwards or from any of positions 188 to 194 onwards. More generally speaking, the skilled person can prepare, for a given combination of a Cas nickase and a deaminase, fusion constructs with both conceivable orientations (nickase N- or C-terminal) and identify the one providing the more desirable editing profile.

This preferred embodiment relates to preferred orientations of specific fusion constructs. It is understood that the term “fused” does not require, but allows for the presence of a linker between the two recited components. In fact, in accordance with the invention, the two components are linked by any one of items (i) to (iii) of the first aspect. It is only item (ii), directed to a direct fusion, which implements the notion of “fused” in a narrow sense, i.e. without any intervening moieties.

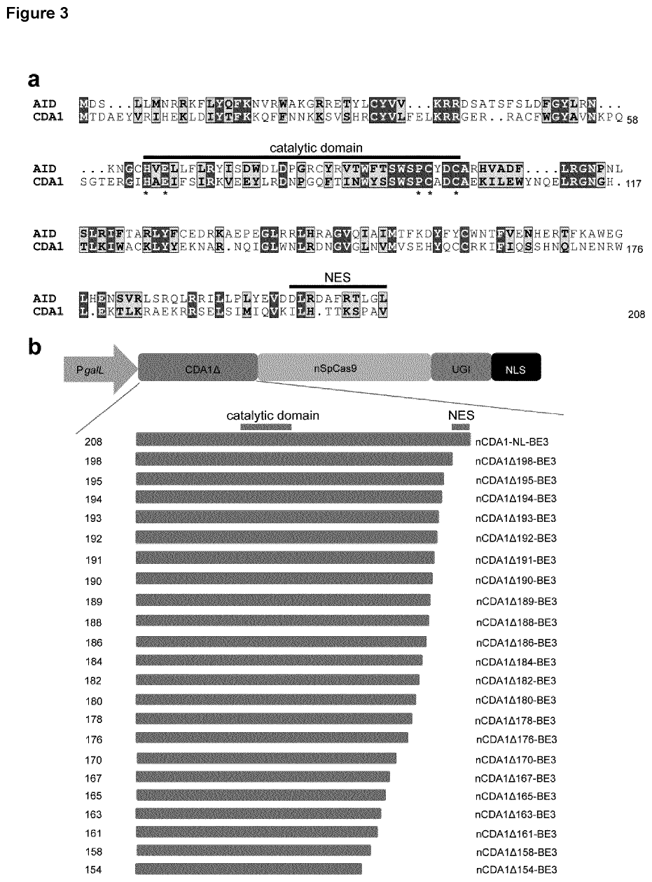

In a further preferred embodiment, said deaminase is CDA1 and wherein the C-terminus of said CDA1 is truncated. Also envisaged is a deaminase consisting of the sequence of SEQ ID NO: 17 or 18; see the highlighted catalytic domains in FIG. 3a.

In a further preferred embodiment, (a) said Cas protein consists of the amino acid sequence of SEQ ID NO: 1 or a sequence with at least 80% identity thereto and preferably providing nickase activity or is encoded by the nucleic acid sequence of SEQ ID NO: 2 or a sequence with at least 80% identity thereto and preferably encoding a protein with nickase activity, and is preferably selected from VQR-Cas9 (amino acid sequence of SEQ ID NO: 121 VRER-Cas9 (amino acid sequence of SEQ ID NO: 122), xCas9 (amino acid sequence of SEQ ID NO: 123), and Cas9-NG (amino acid sequence of SEQ ID NO: 124), or encoded by any one of SEQ ID NOs: 23, 24, 25 or 26; and/or (b) said deaminase consists of the amino acid sequence of any one of SEQ ID NOs: 205, 129, 137, 169, 176, 183, 198, 212, 219, 3 and 5, a sequence with at least 80% identity and providing deaminase activity or a truncated version of any such sequence, or is encoded by the nucleic acid sequence of any one of SEQ ID NOs: 107, 31, 55, 71, 78, 85, 100, 114, 220, 4 and 6, a sequence with at least 80% identity and encoding a protein with deaminase activity or a truncated version of any such sequence.

Any of the Cas proteins in accordance with item (a) of this preferred embodiment can be used to implement the Cas protein component in the base editors given in Table 1 below. Particular preference in that respect is given to the Cas nickases VQR-Cas9, VRER-Cas9, xCas9 and Cas9-NG.

The above disclosed Cas9 variants recognize different protospacer adjacent motifs (PAMs). In particular, VQR-Cas9 recognizes NGA, VRER-Cas9 recognizes NGCG, xCas9 recognizes the PAM sequences NG, GAA and GRT, and Cas9-NG recognizes the shorter PAM sequence which is NG. In all cases N designates any nucleotide. Of particular interest in that respect is the shortened PAM sequence recognized by Cas9-NG, which occurs, on an average basis, with a higher frequency than any trinucleotide sequence.

Preferred is also any level of sequence identity above said at least 80% identity, be it at the amino acid or the nucleic acid level. Accordingly, included are sequence identity levels such as at least 85% identity, at least 90%, at least 91%, at least 92%, at least 93%, at least 94%, at least 95%, at least 96%, at least 97%, at least 98% and at least 99% sequence identity.

The term “truncated version” refers to truncations in accordance with the invention, i.e., truncation of residues which are not essential for catalytic activity. A decrease of catalytic activity, while not being preferred, is acceptable, in particular in case the editing profile is sharpened at the same time. Having said that, Example 2 demonstrates that large truncations in many instances do not entail such decrease.

The amino acid sequence of SEQ ID NO: 1 defines a preferred Cas9 nickase. The amino acid sequences of SEQ ID NOs: 3 and 5 define preferred APOBEC1 and CDA1 deaminases, respectively.

In a further preferred embodiment, said compound is a single polypeptide and comprises or consists of an amino acid sequence selected from SEQ ID NOs: 204, 7, 9, 11, 13, 136, 218, 190, 144, 168, 182, 128, 152, 204 and 211.

The amino acid sequences of SEQ ID NOs: 7 and 9 comprise, from N- to C-terminus, the deaminase of SEQ ID NO: 3, a preferred peptidic linker (SEQ ID NO: 15 in case of SEQ ID NO: 7 and SEQ ID NO: 16 in case of SEQ ID NO: 9) and the Cas9 nickase of SEQ ID NO: 1. Furthermore, they comprise at their respective C-terminus an UGI (SEQ ID NO: 19) and an NLS (SEQ ID NO: 20). In either case, i.e. between the C-terminus of the nickase and the N-terminus of UGI, and furthermore between the C-terminus of UGI and the N-terminus of NLS, a short linker sequence consisting of SGGS (SEQ ID NO: 21) is present.

The sequences of SEQ ID NOs: 11 and 13 relate to particularly preferred base editing compounds of the invention which comprise, form N- to C-terminus, differently truncated versions of the CDA1 deaminase of SEQ ID NO: 5, followed by the Cas9 nickase of SEQ ID NO: 1 an UGI (SEQ ID NO: 19) and an NLS (SEQ ID NO: 20). In either case, i.e. between the C-terminus of the nickase and the N-terminus of UGI, and furthermore between the C-terminus of UGI and the N-terminus of NLS, a short linker sequence consisting of SGGS (SEQ ID NO: 21) is present.

Homologues of the specific UGI of SEQ ID NO: 19 may also be used for the present invention, wherein preferably said homologues exhibit at least 80%, at least 85%, at least 90%, at least 91%, at least 92%, at least 93%, at least 94%, at least 95%, at least 96%, at least 97%, at least 98% and at least 99% sequence identity to the sequence of SEQ ID NO: 19 and furthermore are capable of inhibiting uracil glycosylase.

Similar, homologues of the nuclear localization signal sequence of SEQ ID NO: 20 may be used, wherein preferably said homologues differ by 1, 2 or 3 amino acids from the sequence of SEQ ID NO: 20 and provide for a nuclear localization of the base editor of the invention comprising such sequence.

Particularly preferred base editors of the invention are given in the Table below.

Table 1. Recommendations for BE selection for precision cytosine base editing. The C to be edited is underlined, “no bystander” means absence of other Cs from the activity window of the BEs. N: any nucleotide (including a possible bystander C), D: not C (i.e., A, G or T), R: A or G. “Δ” indicates deletions; e.g. Δ198 means that only residues 1 to 198 are retained; Δ (194-188) means that only residues 188 to 194 are deleted. “n” means that the deaminase is fused to the N-terminus of the Cas protein; and “c” means that the deaminase is fused to the C-terminus of the Cas protein. “NL” means no linker between deaminase and Cas protein. Even though not expressly indicated, all CDA1 deletion variants do not contain a linker; see the corresponding entries of the sequence listing. PAPAPAP is a specific and preferred linker in accordance with the invention. “BE3” as used in this Table designates a class of molecules. It refers to all components of a third generation base editor with all modifications to it being separately indicated. Preferred implementations of the Cas protein are disclosed herein above.

| | | Recommended BEs: each designation refers to |
|---|---|---|
| Distance of | | a genus for exemplary or preferred |
| target | | implementations, a SEQ ID NO is given in |
| C from PAM | Bystander | brackets |
| <−19 | no | nCDA1-BE3 (SEQ ID NO: 136), nCDA1Δ198-BE3 |
| | | (SEQ ID NO: 218), A3A-NL-BE3 (SEQ ID NO: 190) |
| <−19 | no | nCDA1-BE3 (SEQ ID NO: 136), nCDA1Δ198-BE3 |
| | | (SEQ ID NO: 218), A3A-NL-BE3 (SEQ ID NO: 190) |
| | CCDDD | cCDA1-BE3 (SEQ ID NO: 144) |
| −18 | no | nCDA1Δ198-BE3 (SEQ ID NO: 218), n/cCDA1-BE3 |
| | | (SEQ ID NO: 136 or 144), A3A-NL-BE3 (SEQ ID |
| | | NO: 190) |
| | NCN | nCDA1Δ(194-188)-BE3 (SEQ ID NO: 168) |
| −17 | no | nCDA1Δ198-BE3 (SEQ ID NO: 218), cCDA1-BE3 |
| | | (SEQ ID NO: 144), A3A-BE3 (SEQ ID NO: 182), |
| | | nCDA1-BE3 (SEQ ID NO: 136) |
| | DCN | nCDA1Δ(194-188)-BE3 (SEQ ID NO: 168) |
| −16 | no | nCDA1Δ198-BE3 (SEQ ID NO: 218), A3A-BE3 |
| | | (SEQ ID NO: 182) |
| | DDDCC | cCDA1-BE3 (SEQ ID NO: 144) |
| | TCC | cCDA1-BE3 (SEQ ID NO: 144), BE-PAPAPAP |
| | | (SEQ ID NO: 152) |
| | NCD | A3AΔ182-BE3 (SEQ ID NO: 204), |
| | | A3A(Y130F)Δ186-BE3 (SEQ ID NO: 211) |
| −15 | no | nCDA1Δ198-BE3 (SEQ ID NO: 218), A3A-BE3 |
| | | (SEQ ID NO: 182) |
| | CDCD | BE-PAPAPAP (SEQ ID NO: 152), A3AΔ182-BE3 |
| | | (SEQ ID NO: 204), A3A(Y130F)Δ186-BE3 (SEQ ID |
| | | NO: 211) |
| | DCN | A3AΔ182-BE3 (SEQ ID NO: 204), |
| | | A3A(Y130F)Δ186-BE3 (SEQ ID NO: 211) |
| | RCCD | BE-PAPAPAP (SEQ ID NO: 152) |
| −14 | no | A3A-BE3 (SEQ ID NO: 182), nCDA1-BE3 (SEQ ID |
| | | NO: 136) |
| | DDCC | BE-PAPAPAP (SEQ ID NO: 152) |
| >−14 | no | A3A-BE3 (SEQ ID NO: 182), nCDA1-BE3 (SEQ ID |
| | | NO: 136) |

The term “bystander” is used to provide information about the tolerance of the base editor under consideration to the presence of further Cs within the activity window in addition to the specific C to be edited. A higher degree of tolerance in said sense means that even in the presence of further Cs in the proximity of the specific site to be edited, only or substantially only the specific site of interest (location indicated in the first column of Table 1; underlined in second column) is edited. If “no” is given in the bystander column, this means that preferably no further Cs should be in the proximity of the site to be edited if maximum precision is desired. On the other hand, if certain bystanders are given, this means that these positions are not edited or edited only to a low degree, even if they are occupied by Cs or the residues indicated in the Table.

In particularly preferred embodiments, the present invention provides the following uses:

Use of a polypeptide comprising or consisting of the sequence of any one of 204, 136, 218, 190, 144, 168, 182, 128, 152, 204 and 211 as base editor. Preferably, and this applies generally, said base editors convert a C into a T. As disclosed herein above, different Cas proteins, and different modified versions of the same Cas protein recognize different protospacer adjacent motifs (PAMs). The specific PAM sequences recognized by a given Cas protein are given further above.

Use of a polypeptide comprising or consisting of the sequence of SEQ ID NO: 136 for editing a base which is more than 19 bases upstream from the protospacer adjacent motif (PAM). Preferably, said polypeptide consists of said sequence.

Use of a polypeptide comprising or consisting of the sequence of SEQ ID NO: 218 for editing a base which is more than 19 bases upstream from the protospacer adjacent motif (PAM). Preferably, said polypeptide consists of said sequence.

Use of a polypeptide comprising or consisting of the sequence of SEQ ID NO: 190 for editing a base which is more than 19 bases upstream from the protospacer adjacent motif (PAM). Preferably, said polypeptide consists of said sequence.

Use of a polypeptide comprising or consisting of the sequence of SEQ ID NO: 136 for editing a base which is 19 bases upstream from the protospacer adjacent motif (PAM). Preferably, said polypeptide consists of said sequence.

Use of a polypeptide comprising or consisting of the sequence of SEQ ID NO: 218 for editing a base which is 19 bases upstream from the protospacer adjacent motif (PAM). Preferably, said polypeptide consists of said sequence.

Use of a polypeptide comprising or consisting of the sequence of SEQ ID NO: 190 for editing a base which is 19 bases upstream from the protospacer adjacent motif (PAM). Preferably, said polypeptide consists of said sequence.

Use of a polypeptide comprising or consisting of the sequence of SEQ ID NO: 144 for editing a base which is 19 bases upstream from the protospacer adjacent motif (PAM). Preferably, said polypeptide consists of said sequence.

Use of a polypeptide comprising or consisting of the sequence of SEQ ID NO: 218 for editing a base which is 18 bases upstream from the protospacer adjacent motif (PAM). Preferably, said polypeptide consists of said sequence.

Use of a polypeptide comprising or consisting of the sequence of SEQ ID NO: 136 or 144 for editing a base which is 18 bases upstream from the protospacer adjacent motif (PAM). Preferably, said polypeptide consists of said sequence.

Use of a polypeptide comprising or consisting of the sequence of SEQ ID NO: 190 for editing a base which is 18 bases upstream from the protospacer adjacent motif (PAM). Preferably, said polypeptide consists of said sequence.

Use of a polypeptide comprising or consisting of the sequence of SEQ ID NO: 168 for editing a base which is 18 bases upstream from the protospacer adjacent motif (PAM). Preferably, said polypeptide consists of said sequence.

Use of a polypeptide comprising or consisting of the sequence of SEQ ID NO: 218 for editing a base which is 17 bases upstream from the protospacer adjacent motif (PAM). Preferably, said polypeptide consists of said sequence.

Use of a polypeptide comprising or consisting of the sequence of SEQ ID NO: 144 for editing a base which is 17 bases upstream from the protospacer adjacent motif (PAM). Preferably, said polypeptide consists of said sequence.

Use of a polypeptide comprising or consisting of the sequence of SEQ ID NO: 182 for editing a base which is 17 bases upstream from the protospacer adjacent motif (PAM). Preferably, said polypeptide consists of said sequence.

Use of a polypeptide comprising or consisting of the sequence of SEQ ID NO: 136 for editing a base which is 17 bases upstream from the protospacer adjacent motif (PAM). Preferably, said polypeptide consists of said sequence.

Use of a polypeptide comprising or consisting of the sequence of SEQ ID NO: 128 for editing a base which is more than 17 bases upstream from the protospacer adjacent motif (PAM). Preferably, said polypeptide consists of said sequence.

Use of a polypeptide comprising or consisting of the sequence of SEQ ID NO: 168 for editing a base which is 17 bases upstream from the protospacer adjacent motif (PAM). Preferably, said polypeptide consists of said sequence.

Use of a polypeptide comprising or consisting of the sequence of SEQ ID NO: 128 for editing a base which is 16 bases upstream from the protospacer adjacent motif (PAM). Preferably, said polypeptide consists of said sequence.

Use of a polypeptide comprising or consisting of the sequence of SEQ ID NO: 128 for editing a base which is 16 bases upstream from the protospacer adjacent motif (PAM). Preferably, said polypeptide consists of said sequence.

Use of a polypeptide comprising or consisting of the sequence of SEQ ID NO: 182 for editing a base which is 16 bases upstream from the protospacer adjacent motif (PAM). Preferably, said polypeptide consists of said sequence.

Use of a polypeptide comprising or consisting of the sequence of SEQ ID NO: 144 for editing a base which is 16 bases upstream from the protospacer adjacent motif (PAM). Preferably, said polypeptide consists of said sequence.

Use of a polypeptide comprising or consisting of the sequence of SEQ ID NO: 152 for editing a base which is 16 bases upstream from the protospacer adjacent motif (PAM). Preferably, said polypeptide consists of said sequence.

Use of a polypeptide comprising or consisting of the sequence of SEQ ID NO: 204 for editing a base which is 16 bases upstream from the protospacer adjacent motif (PAM). Preferably, said polypeptide consists of said sequence.

Use of a polypeptide comprising or consisting of the sequence of SEQ ID NO: 211 for editing a base which is 16 bases upstream from the protospacer adjacent motif (PAM). Preferably, said polypeptide consists of said sequence.

Use of a polypeptide comprising or consisting of the sequence of SEQ ID NO: 128 for editing a base which is 15 bases upstream from the protospacer adjacent motif (PAM). Preferably, said polypeptide consists of said sequence.

Use of a polypeptide comprising or consisting of the sequence of SEQ ID NO: 218 for editing a base which is 15 bases upstream from the protospacer adjacent motif (PAM). Preferably, said polypeptide consists of said sequence.

Use of a polypeptide comprising or consisting of the sequence of SEQ ID NO: 182 for editing a base which is 15 bases upstream from the protospacer adjacent motif (PAM). Preferably, said polypeptide consists of said sequence.

Use of a polypeptide comprising or consisting of the sequence of SEQ ID NO: 152 for editing a base which is 15 bases upstream from the protospacer adjacent motif (PAM). Preferably, said polypeptide consists of said sequence.

Use of a polypeptide comprising or consisting of the sequence of SEQ ID NO: 204 for editing a base which is 15 bases upstream from the protospacer adjacent motif (PAM). Preferably, said polypeptide consists of said sequence.

Use of a polypeptide comprising or consisting of the sequence of SEQ ID NO: 211 for editing a base which is 15 bases upstream from the protospacer adjacent motif (PAM). Preferably, said polypeptide consists of said sequence.

Use of a polypeptide comprising or consisting of the sequence of SEQ ID NO: 211 for editing a base which is 14 bases upstream from the protospacer adjacent motif (PAM). Preferably, said polypeptide consists of said sequence.

Use of a polypeptide comprising or consisting of the sequence of SEQ ID NO: 182 for editing a base which is 14 bases upstream from the protospacer adjacent motif (PAM). Preferably, said polypeptide consists of said sequence.

Use of a polypeptide comprising or consisting of the sequence of SEQ ID NO: 136 for editing a base which is 14 bases upstream from the protospacer adjacent motif (PAM). Preferably, said polypeptide consists of said sequence.

Use of a polypeptide comprising or consisting of the sequence of SEQ ID NO: 152 for editing a base which is 14 bases upstream from the protospacer adjacent motif (PAM). Preferably, said polypeptide consists of said sequence.

Use of a polypeptide comprising or consisting of the sequence of SEQ ID NO: 182 for editing a base which is less than 14 bases upstream from the protospacer adjacent motif (PAM). Preferably, said polypeptide consists of said sequence.

Use of a polypeptide comprising or consisting of the sequence of SEQ ID NO: 136 for editing a base which is less than 14 bases upstream from the protospacer adjacent motif (PAM). Preferably, said polypeptide consists of said sequence.

Use of a polypeptide comprising or consisting of the sequence of SEQ ID NO: 128 for editing a base which is less than 14 bases upstream from the protospacer adjacent motif (PAM). Preferably, said polypeptide consists of said sequence.

As can be seen from these preferred aspects of the invention, the design approach chosen by the inventors greatly amplifies the spectrum of available base editors. This does not only apply to the PAM sequence being recognized, but also to the distance of the residue to be edited from said PAM sequence.

In a second aspect, the present invention relates to a nucleic acid encoding the compound of any one of the preceding claims, to the extent said compound is a single polypeptide.

A nucleic acid encoding a particular amino acid sequence may consist of or comprise the particular nucleic acid sequence encoding the amino acid sequence. Nucleic acids in accordance with the second aspect embrace either genus, i.e., flanking nucleotide sequences may be present but do not have to be.

Preferred flanking sequences include, for example, a promoter at the 5′-end and/or a terminator, preferably with a polyadenylation signal, at the 3′ end. Suitable promoters and terminators are at the skilled person's disposal.

It is understood that any preferred embodiment of the base editing compound in accordance with the first aspect of the invention gives rise to preferred embodiments of the nucleic acid of the second aspect, to the extent applicable.

Similarly, and even if certain claims might be limited to certain back-references, it is understood that any preferred embodiments of the base editing compound in accordance with the invention may be combined such that such combined subject-matter is also embraced by the present invention. Furthermore, any such subject-matter is also a preferred implementation of any of the further aspects of the invention disclosed further below.

In a third aspect, the present invention provides a method of base editing, said method comprising introducing into a cell a nucleic acid in accordance with the second aspect or a compound in accordance with the first aspect.

In a preferred embodiment, said method further comprises introducing into said cell a guide nucleic acid for said Cas protein, preferably said Cas nickase or said dead Cas.

It is a known property of Cas proteins that their capability to target a particular region on a target nucleic acid, said target nucleic acid preferably being DNA or RNA, is conferred by a guide nucleic acid, preferably guide RNA. Accordingly, in accordance with this preferred embodiment, such guide nucleic acid is to be introduced into the cell to which a compound or a nucleic acid of the second aspect is to be introduced. Introducing of the base editing compound or nucleic acid in accordance with the third aspect on the one hand and of the guide nucleic acid on the other hand may be performed concomitantly or in any order.

The base editors of the present invention are widely applicable in both prokaryotic and eukaryotic organisms and cells. For the purpose of introducing said base editor or nucleic acids encoding it into said organisms or cells, any of the art-established methods of transducing, transforming or transfecting can be used. Suitable methods can be chosen by the skilled person without further ado.

In accordance with established knowledge about the CRISPR/Cas system, it is furthermore understood that such guide nucleic acids are chosen in a manner which target the compound of the invention to a site in the target nucleic acid which furthermore comprises a protospacer adjacent motif (PAM) which is recognized by the particular Cas-variant to be used, preferred Cas-variants being Cas9 and Cas12. Preferred PAMs include those known in the art such as 5′-NGG-3′ in case of Cas9, wherein N can be any nucleobase. Cas12 usually recognizes a PAM motif which is rich in T (e.g., 5′-TTN-3′, N being any nucleobase). Further PAM sequences and Cas proteins recognizing them are disclosed herein further above.

In a further preferred embodiment, said method is performed in vitro or ex vivo.

Also preferred are applications of said method in the fields of plant breeding and agricultural biotechnology. In such a context, base editing may serve to modify the genotype of plants such that useful traits are generated or enhanced, useful traits including resistances, stress tolerance, yield and food quality.

In a fourth aspect, the present invention provides a pharmaceutical composition comprising or consisting of (a) the compound in accordance with the second aspect; and/or (b) the nucleic acid in accordance with the first aspect.

In a preferred embodiment, said pharmaceutical composition further comprises or further consists of a guide nucleic acid for said Cas protein, wherein said guide nucleic acid comprises a sequence which is homologous to a subsequence of a target gene, wherein said target gene is associated with a genetic disorder.

Preferred genetic disorders are those which arise from a point mutation or SNP. To explain further, in such a setting, the beneficial properties of the present invention, namely the precise editing profile is highly desirable and advantageous.

Pharmaceutical compositions in accordance with the present invention may comprise further active agents. It is preferred, though, that the recited agents, namely compound of the invention, nucleic acid of the second aspect and the optional guide nucleic acid are the only pharmaceutically active agents.

As is well-established in the art, pharmaceutical compositions may comprise excipients, fillers and/or diluents. Examples of suitable pharmaceutical carriers, excipients and/or diluents are well known in the art and include phosphate buffered saline solutions, water, emulsions, such as oil/water emulsions, various types of wetting agents, sterile solutions etc. Compositions comprising such carriers can be formulated by well known conventional methods. These pharmaceutical compositions can be administered to the subject at a suitable dose. Administration of the suitable compositions may be effected by different ways, e.g., by intravenous, intraperitoneal, subcutaneous, intramuscular, topical, intradermal, intranasal or intrabronchial administration.

The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. Proteinaceous pharmaceutically active matter may be present in amounts between 1 ng and 10 mg/kg body weight per dose; however, doses below or above this exemplary range are envisioned, especially considering the aforementioned factors. If the regimen is a continuous infusion, it should also be in the range of 1 μg to 10 mg units per kilogram of body weight per minute.

In a fifth aspect, the present invention provides a compound in accordance with the first aspect or a nucleic acid in accordance with the second aspect, and a guide nucleic acid for said nickase for use in a method of treating, alleviating or preventing a disorder, wherein said guide nucleic acid comprises a sequence which is homologous to a subsequence of a target gene, wherein said disorder is associated with a point mutation or an SNP in said target gene.

In a sixth aspect, the present invention provides a kit comprising or consisting of (a)(i) one or more compounds in accordance with the second aspect; and/or (ii) one or more nucleic acids in accordance with the first aspect.

In a preferred embodiment, said kit furthermore comprises or further consists of (b) one or more guide nucleic acids for the nickase comprised in said compound, wherein each of said guide nucleic acids comprises a sequence which is identical to a subsequence of a given target gene; and/or (c) a manual comprising instructions for performing the method of the third aspect.

In a further preferred embodiment, said kit comprises a plurality of said compounds and/or a plurality of said nucleic acids, wherein at least two of said compounds of (a)(i) or at least two of the compounds encoded by said nucleic acids of (a)(ii) differ with regard to their base editing profile. Preferably, the difference with regard to their base editing profile is the distance of the edited position from the PAM motif.

As can be seen from the evidence comprised in Example 2, the two different strategies in accordance with the present invention, i.e. deaminase truncation and use of a rigid and/or short linker, provide in either case for very localized editing profiles on the target nucleic acid, wherein the specific location which is edited is located upstream from the PAM motif to different extents depending on the strategy chosen and/or the specific implementation for a given strategy.

To explain further, the particularly preferred base editor in accordance with the present invention which has the amino acid sequence of SEQ ID NO: 9 (also designated BE-PAPAPAP herein) mainly edits within an activity window from −14 to −16. This window size generally does not amount to a deficiency. For example, this base editor may be used in combination with a guide sequence which targets the compound of the invention to a region within the target nucleic acid which has exactly one cytidine within said activity window.

Base editors with CDA1 truncations such as the particularly preferred base editors having the amino acid sequences of SEQ ID NOs: 11 and 13 mainly edit at position −18.

As explained above, the present invention furthermore envisages the use of distinct Cas-derived targeting components or Cas proteins, in particular Cas nickases, e.g., derived from Cas9 or Cas12. These in turn have different preferences with regard to the PAM motifs.

Before such background, kits in accordance with the invention are provided which offer a plurality of base editors which differ from each other with regard to the distance of the edited position from a PAM motif. As such, provided is a versatile toolkit which allows highly targeted intervention at a plurality of sites upstream of a given PAM motif.

In a seventh aspect, the present invention provides the use of a peptide as defined in any one of the preceding claims or of a non-peptidic linker as defined in relation to the first aspect for covalently connecting a Cas protein such as a Cas nickase (nCas) or a dead Cas (dCas) and a deaminase (DA) to provide a base editing compound.

In a preferred embodiment, said deaminase is truncated at the N- or C-terminus.

Again, such truncations are truncations in the sense of the present disclosure, i.e. truncations which do not significantly affect enzymatic activity of said deaminase.

As regards the embodiments characterized in this specification, in particular in the claims, it is intended that each embodiment mentioned in a dependent claim is combined with each embodiment of each claim (independent or dependent) said dependent claim depends from. For example, in case of an independent claim 1 reciting 3 alternatives A, B and C, a dependent claim 2 reciting 3 alternatives D, E and F and a claim 3 depending from claims 1 and 2 and reciting 3 alternatives G, H and I, it is to be understood that the specification unambiguously discloses embodiments corresponding to combinations A, D, G; A, D, H; A, D, I; A, E, G; A, E, H; A, E, I; A, F, G; A, F, H; A, F, I; B, D, G; B, D, H; B, D, I; B, E, G; B, E, H; B, E, I; B, F, G; B, F, H; B, F, I; C, D, G; C, D, H; C, D, I; C, E, G; C, E, H; C, E, I; C, F, G; C, F, H; C, F, I, unless specifically mentioned otherwise.

Similarly, and also in those cases where independent and/or dependent claims do not recite alternatives, it is understood that if dependent claims refer back to a plurality of preceding claims, any combination of subject-matter covered thereby is considered to be explicitly disclosed. For example, in case of an independent claim 1, a dependent claim 2 referring back to claim 1, and a dependent claim 3 referring back to both claims 2 and 1, it follows that the combination of the subject-matter of claims 3 and 1 is clearly and unambiguously disclosed as is the combination of the subject-matter of claims 3, 2 and 1. In case a further dependent claim 4 is present which refers to any one of claims 1 to 3, it follows that the combination of the subject-matter of claims 4 and 1, of claims 4, 2 and 1, of claims 4, 3 and 1, as well as of claims 4, 3, 2 and 1 is clearly and unambiguously disclosed.

The figures show:

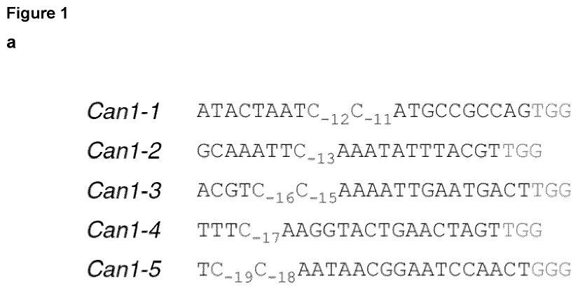

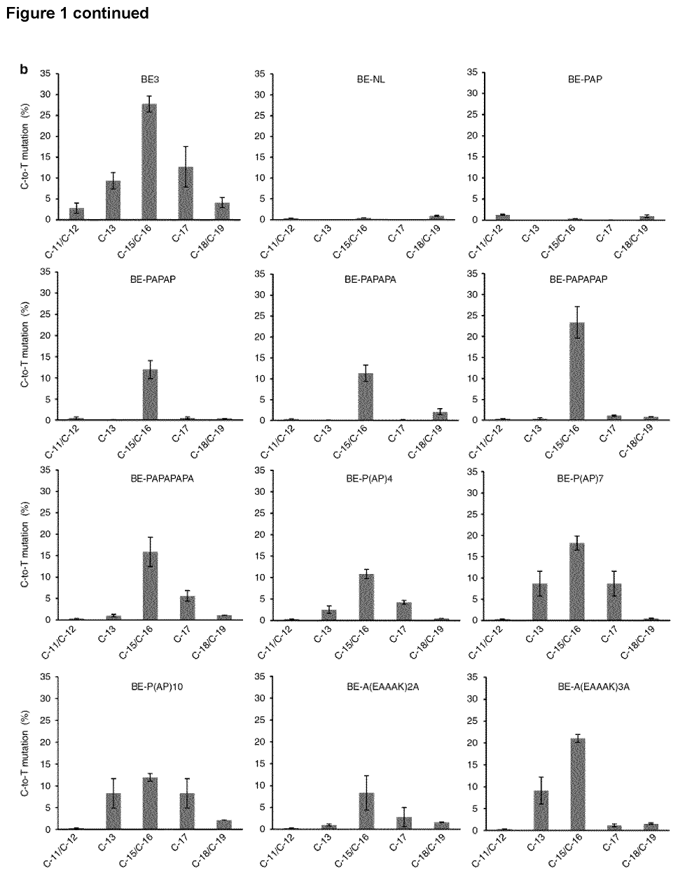

FIG. 1. Rigid linkers narrow the width of the editing window of BE3. a Protospacers and PAM (blue; C-terminal 3 nt) sequences of the genomic loci tested, with the target Cs shown in red (with subscripts indicating the respective position). Subscript numbers indicate the positions of the cytidines relative to the PAM. C-to-T editing at any of the indicated Cs inactivates the Can1 transporter and thus causes resistance to canavanine (Nishida, K. et al. Targeted nucleotide editing using hybrid prokaryotic and vertebrate adaptive immune systems. Science 353, 1248 (2016)). b Editing efficiency and specificity of the base editors tested as determined by canavanine selection. The x-axis represents the target Cs within the protospacers. The y-axis shows their C-to-T editing frequency (see Example 1). Values and error bars represent the mean and standard deviation of three independent biological replicates.

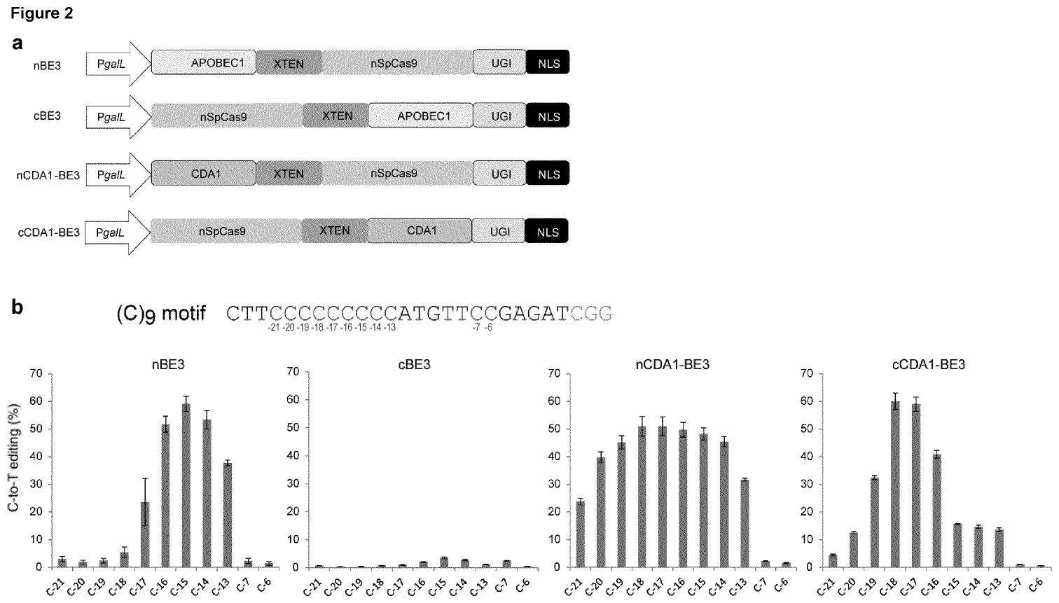

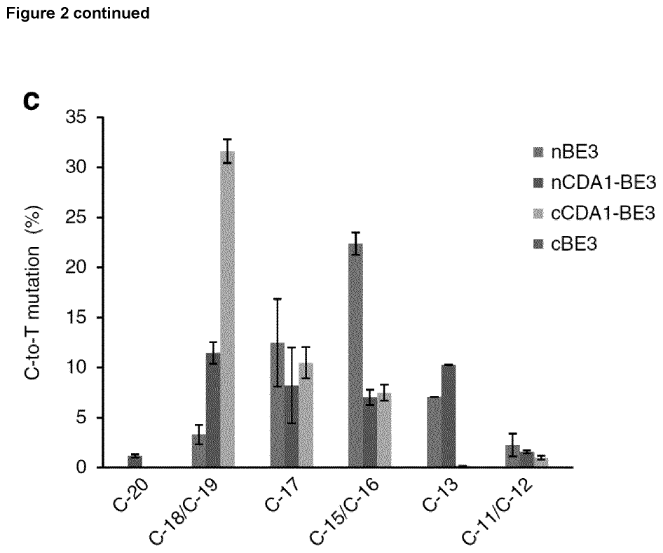

FIG. 2. Comparison of N- and C-terminal deaminase fusions to nCas9. a Structure of nBE3 (=BE3; (Komor, A. C., Kim, Y. B., Packer, M. S., Zuris, J. A. & Liu, D. R. Programmable editing of a target base in genomic DNA without doublestranded DNA cleavage. Nature 533, 420-424 (2016))), cBE3, nCDA1-BE3, and cCDA1-BE3 driven by the GalL inducible promoter. In all constructs, the XTEN linker separates the nucleoside deaminase domain from the nCas9 domain. nSpCas9: Streptococcus pyogenes Cas9 nickase. b Base editors with the deaminase at the N-terminus show broadened base editing windows. The sequence of the target (C)₉ motif is shown with the numbers representing the position of possible editing targets (grey, in the middle of the sequences) relative to the PAM (grey, at the end of the sequences). % of C-to-T editing represents the percentage of total sequencing reads with the target C converted to T. c Base editing outcome of nBE3, cBE3, nCDA1-BE3, and cCDA1-BE3 targeting several sites containing target Cs at different positions (indicated on the x-axis) in the Can1 gene. Values and error bars represent the mean and standard deviation of three independent biological replicates. Order in the legend (top to bottom) corresponds to the order of the bars in the figure (left to right).

FIG. 3. Design of base editors with truncated CDA1 domains. a Amino acid sequence alignment of CDA1 and human AID. The catalytic domain HxE-PCxxC and the nuclear export signal (NES) are indicated by black horizontal lines. The alignment was created by CLUSTALW (Larkin, M. A. et al. Clustal W and Clustal X version 2.0. Bioinformatics 23, 2947-2948 (2007); https://www.genome.jp/tools-bin/clustalw) and graphically formatted with the help of the ESPript 3.0 server (Robert, X. & Gouet, P. Deciphering key features in protein structures with the new ENDscript server. Nucleic Acids Res. 42, W320-W324 (2014).) (http://espript.ibcp.fr/ESPript/ESPript/). Identical amino acid residues are shaded in red (dark grey), similar residues in yellow (light grey). b Schematic representation of base editors with C-terminal CDA1 truncations (named after the last CDA1 residue included).

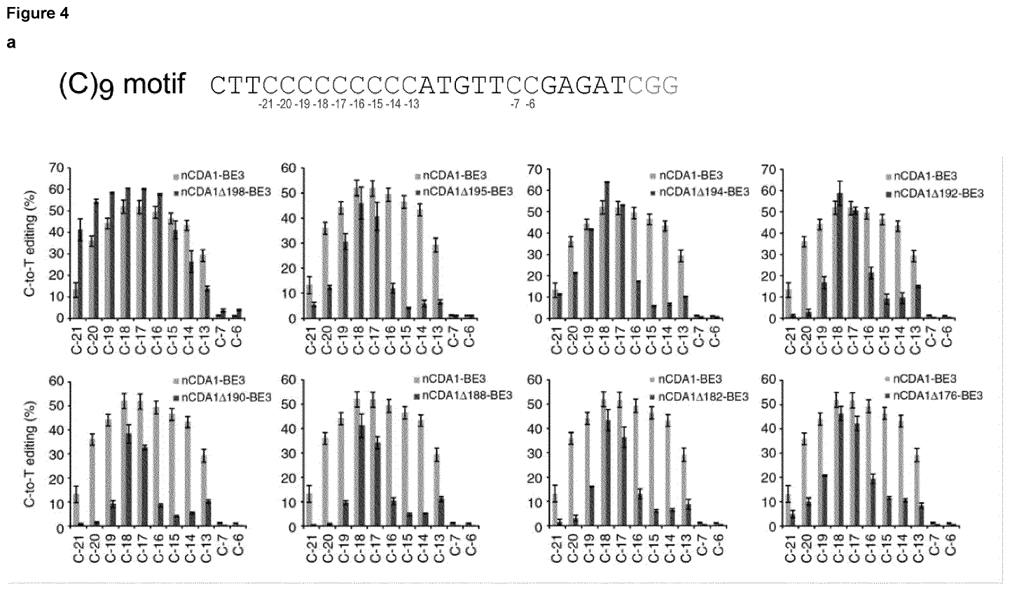

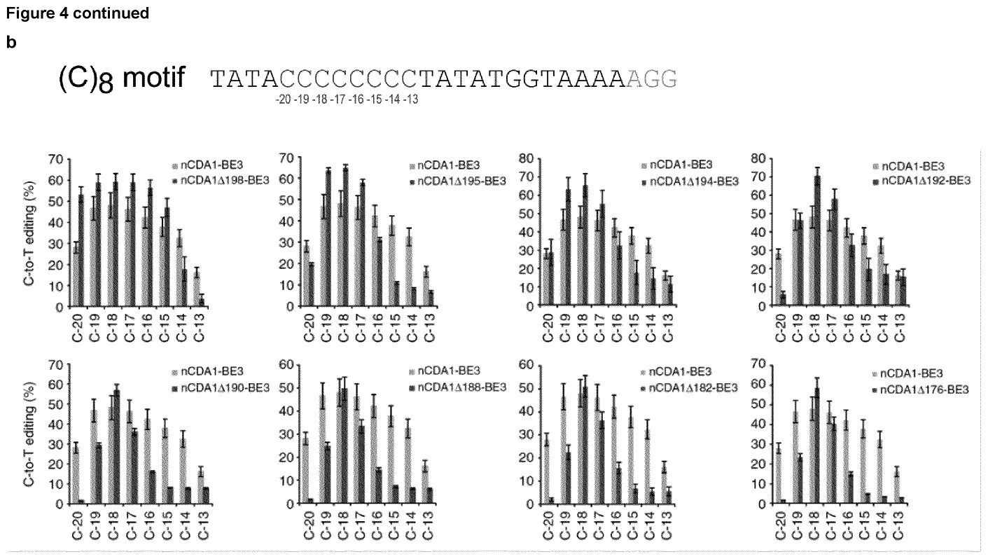

FIG. 4. Effects of C-terminal truncations of the CDA1 domain on the width of the editing window of nCDA1-BE3 base editors. All base editor variants were tested on both (C)₈ (a) and (C)₉ (b) motifs (see Methods). Cs within each target region are shown in red (grey, in the middle of the sequences), with the number below indicating their distance from the PAM (blue; grey, at the end of the sequences). The C-to-T conversion efficiencies are plotted for all Cs within the protospacer, and shown in comparison to the nCDA1-BE3 base editor with the full-length CDA1 (light grey bars). Values and error bars represent the mean and standard deviation of three biological replicates.

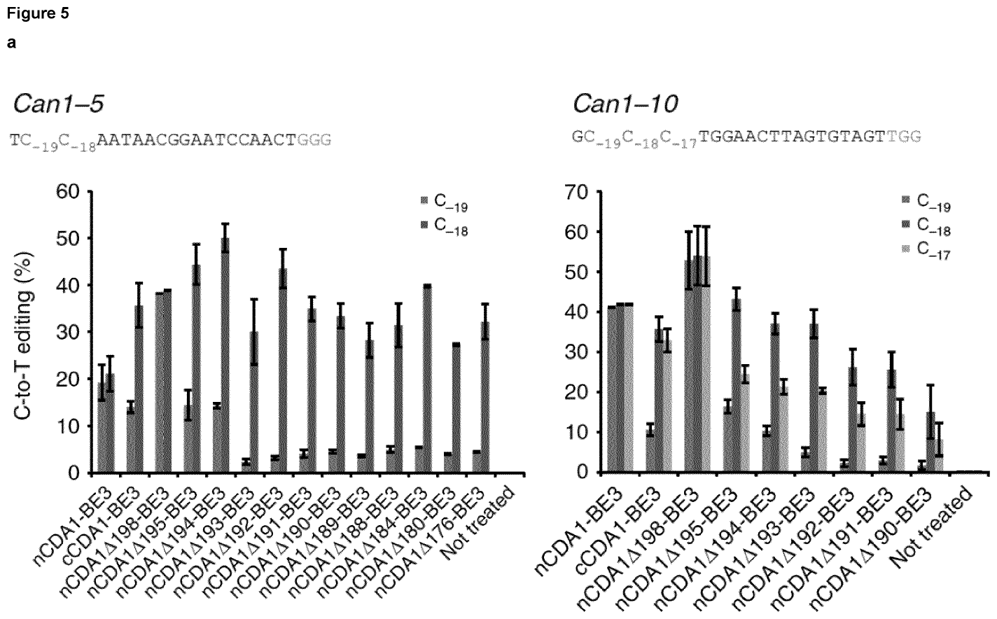

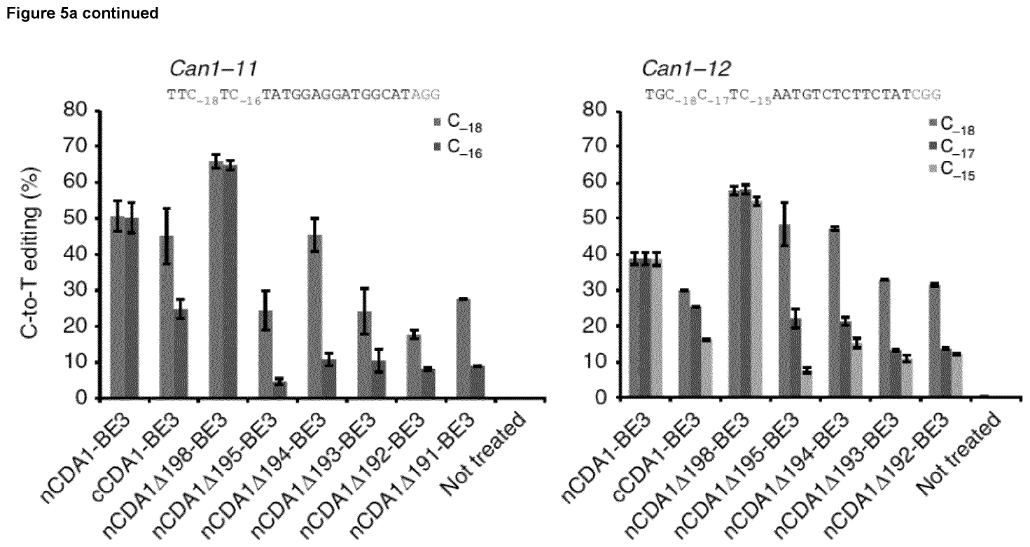

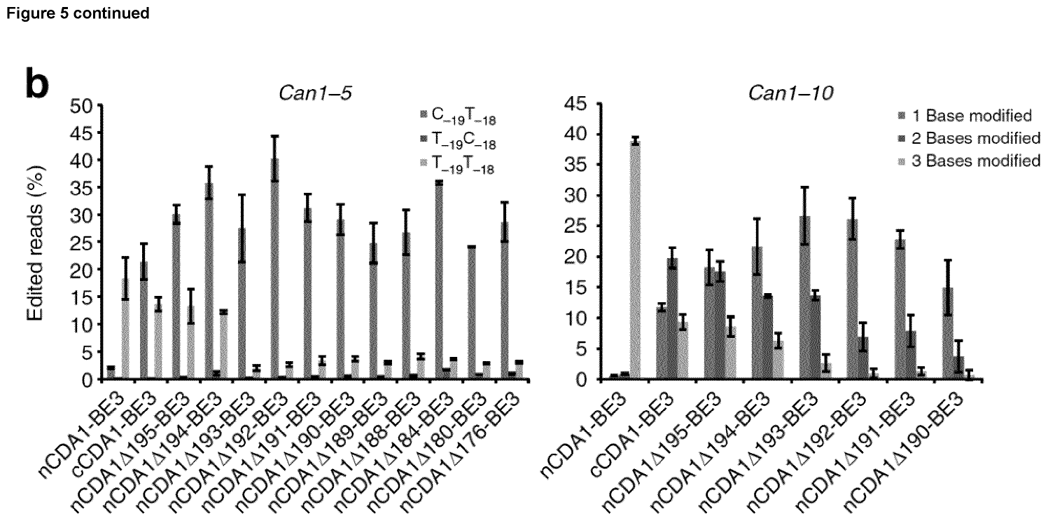

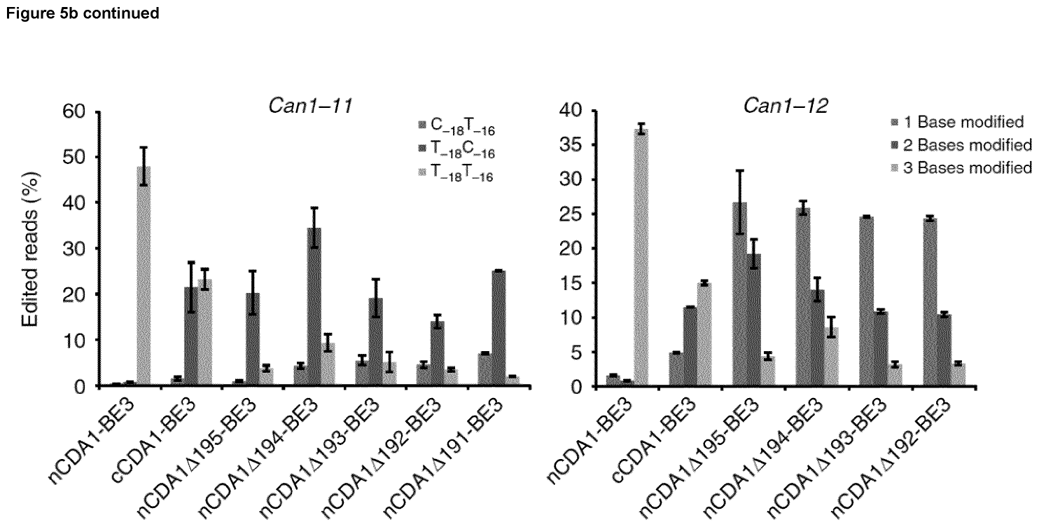

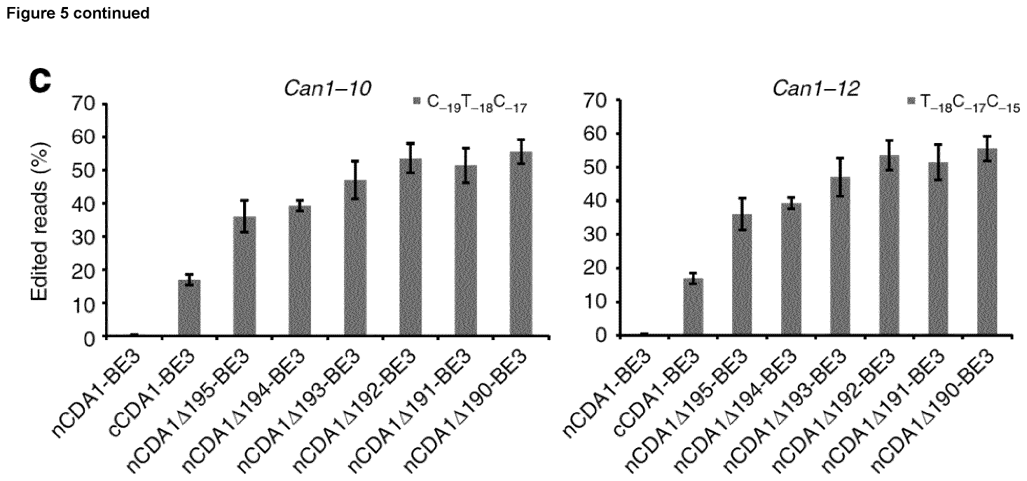

FIG. 5. Base editors with C-terminally truncated CDA1 domains edit position C₋₁₈ with high precision. nCDA1-BE3, cCDA1-BE3, and selected base editors with C-terminally truncated CDA1 domains are compared. a Editing of genomic loci containing multiple cytidines directly adjacent or in close proximity to C₋₁₈. Cytidines representing possible editing targets are shown in red (grey where reproduced in greyscale; with subscripts indicating the respective position) with the subscript number representing their position relative to the PAM (CGG). b, c Base editors with truncated CDA1 domains greatly improve editing product distribution and produce predominantly singly C₋₁₈-modified products. % of edited reads represents the percentage of total sequencing reads containing the products shown. Values and error bars represent the mean and standard deviation of three biological replicates.

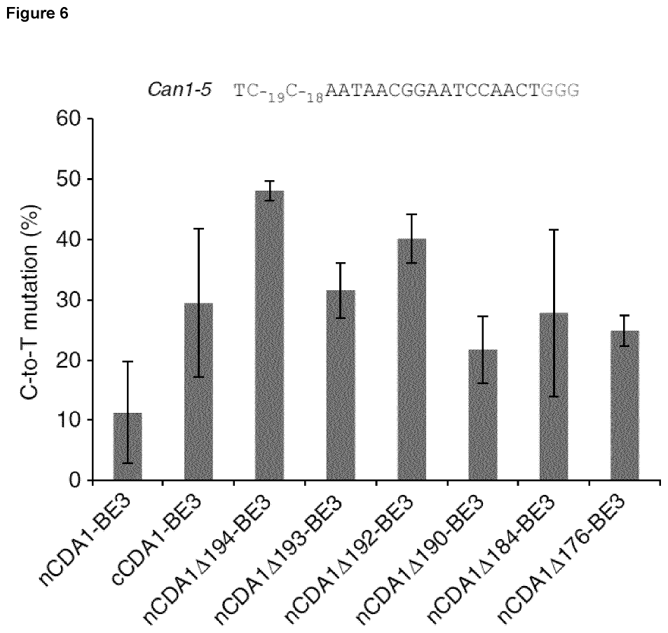

FIG. 6. Analysis of base editing patterns and efficiencies in single yeast colonies selected for canavanine resistance. A comparison of base editing frequencies for nCDA1-BE3, cCDA1-BE3, and selected base editors with truncated CDA1 domains is shown. Yeast cells were transformed with plasmids expressing the base editor and an sgRNA targeting the Can1-5 site. The target sequence is shown with the cytidines that can potentially undergo editing in red (grey, in the middle of the sequences) and the PAM in blue (grey, at the end of the sequences). If C-to-T conversion occurs at position −18 or −19 or both, the Can1 gene will be inactivated and the cell becomes resistant to canavanine. Values and error bars reflect the mean and standard deviation of three biological replicates. See also Table 1.

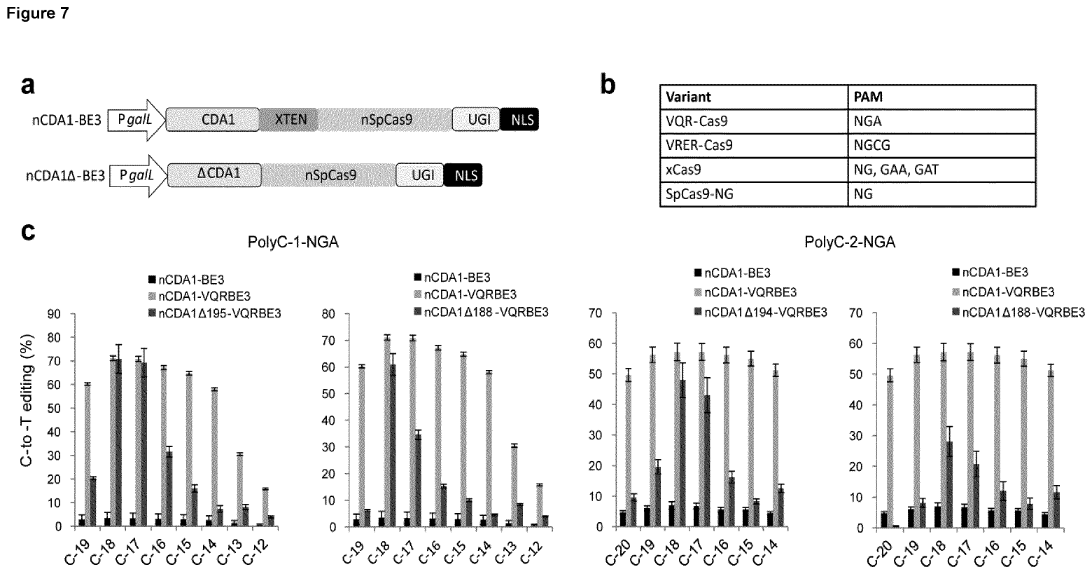

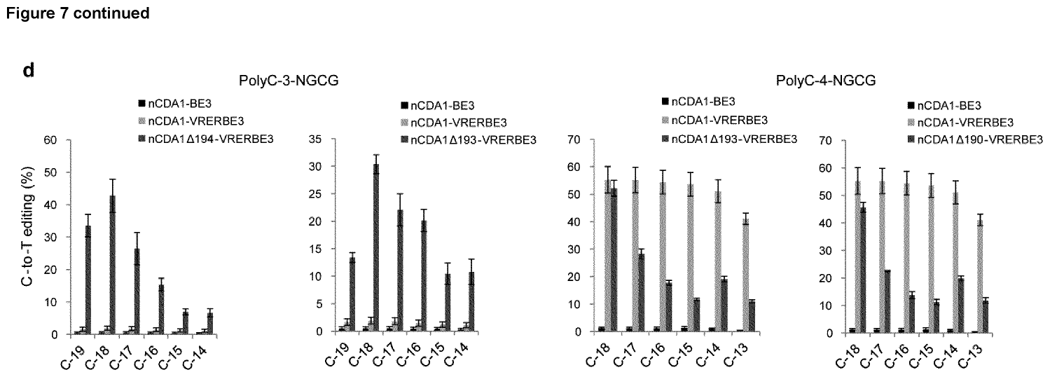

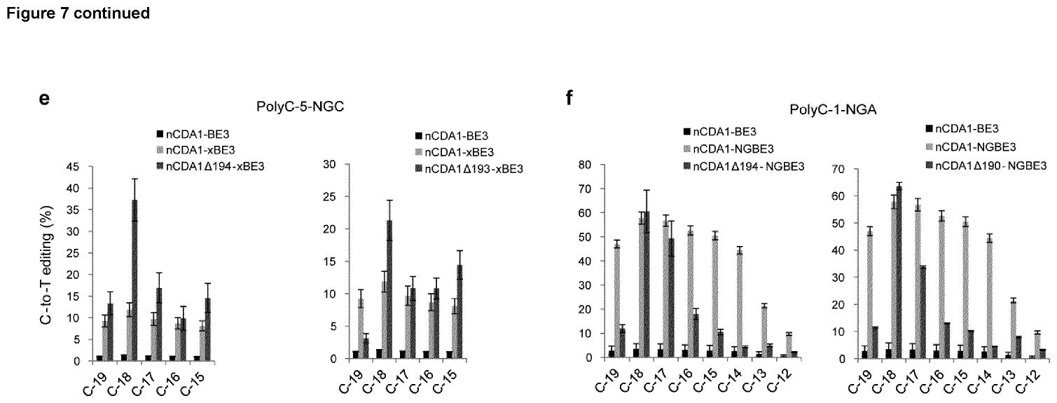

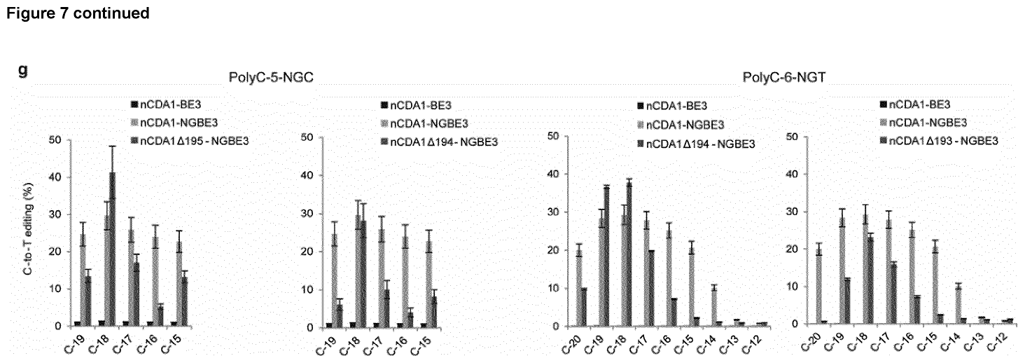

FIG. 7. High-precision base editing at target sites containing non-NGG PAMs. a Structure of nCDA1-BE3 in comparison to base editors harboring CDA1 truncations (ΔCDA1). nSpCas9: Streptococcus pyogenes Cas9 nickase; XTEN: synthetic linker sequence (13); UGI: uracil DNA glycosylase inhibitor; NLS: nuclear localization signal. b Cas9 variants with altered PAM specificities. c-g BE variants with CDA1 truncations mediate high-precision base editing at target sites comprised of multiple cytidines (polyC targets). The x-axis shows the Cs in the target sequence with their position relative to the PAM indicated. The y-axis (C-to-T editing in %) represents the percentage of total sequencing reads with the target C converted to T. Values and error bars represent the mean and standard deviation of three independent biological replicates. c Analysis of base editing precision of VQR-Cas9 BEs fused to selected C-terminally truncated versions of CDA1. For comparison, the BE carrying the full-length CDA1 and the nCDA1-BE3 editor are also included. d Analysis of base editing precision of VRER-Cas9 BEs fused to C-terminally truncated CDA1 versions. e Analysis of base editing precision of xCas9 BEs fused to C-terminally truncated CDA1 versions. f,g Analysis of base editing precision of SpCas9-NG BEs fused to C-terminally truncated CDA1 versions.

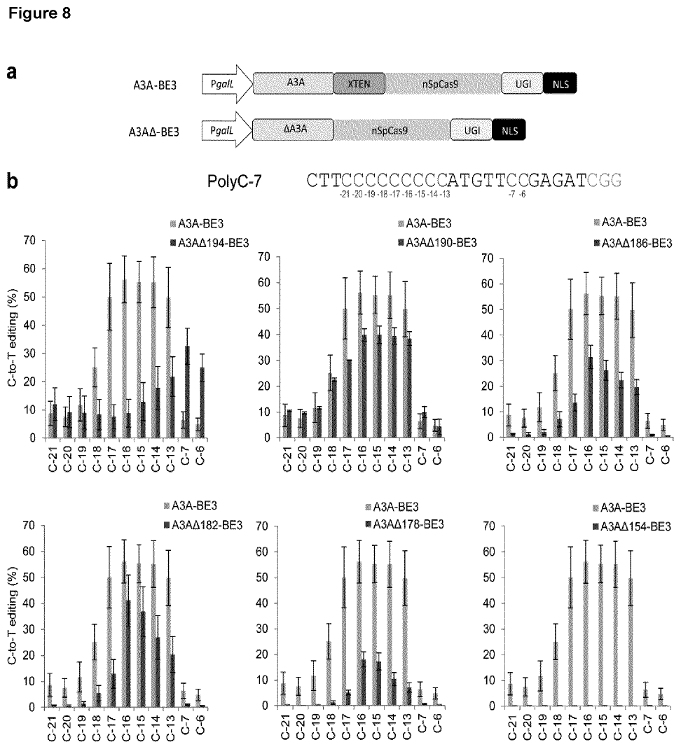

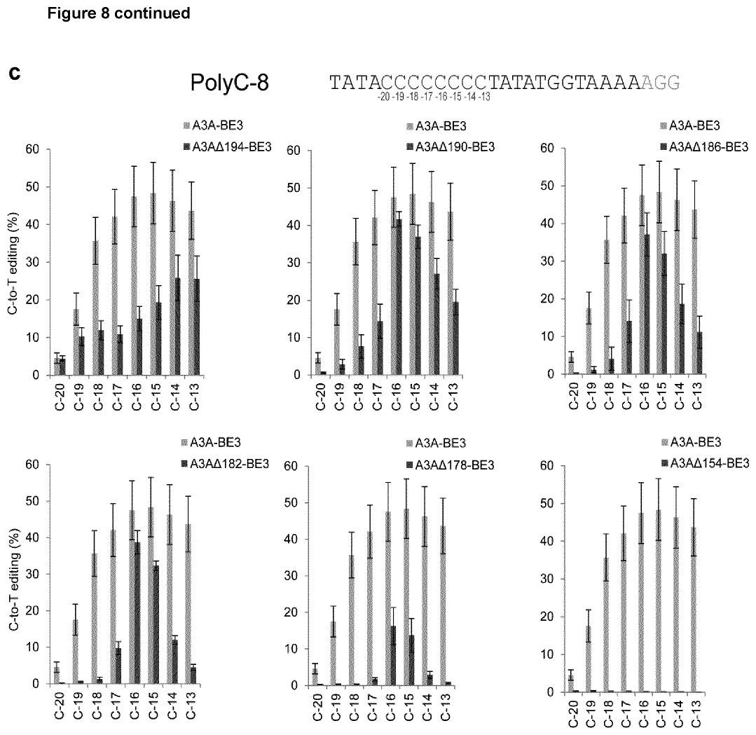

FIG. 8. Base editors with C-terminally truncated A3A sequences exhibit narrowed editing windows. a Structure of A3A-BE3 and BEs with A3A truncations (A3AΔ-BE3 variants). b, c Effects of C-terminal truncations of the A3A domain on the width of the editing window of A3AΔ-BE3s. All base editor variants were tested on both the polyC-7 (b) and polyC-8 (c) sites (see Methods). Cs within each target region are indicated in (grey, in the middle of the sequences), with the number below indicating their distance from the PAM (grey, at the end of the sequences). The C-to-T conversion efficiencies are plotted for all Cs within the protospacer, and shown in comparison to the A3A-BE3 base editor with the full-length A3A (light grey bars). Values and error bars represent the mean and standard deviation of three biological replicates.

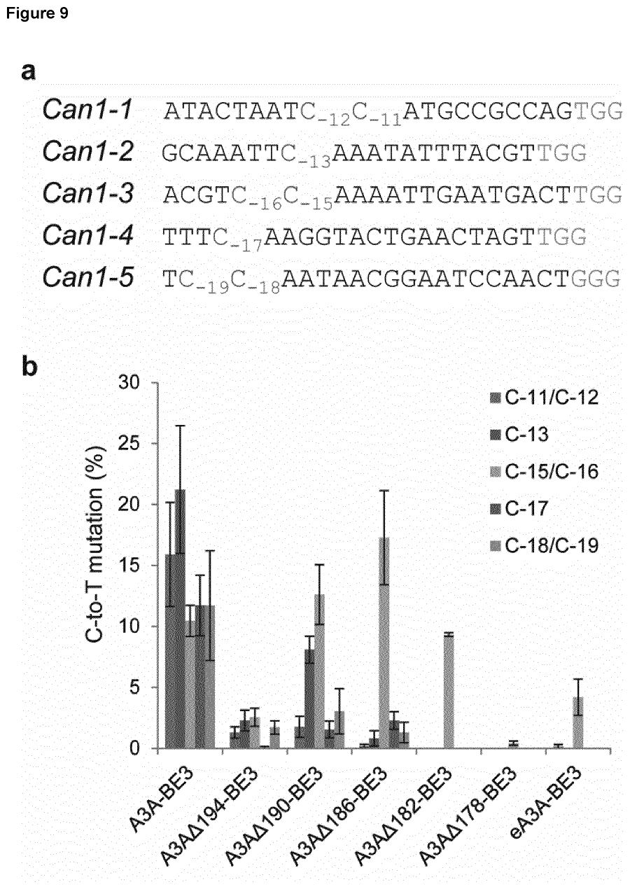

FIG. 9. Base editing outcomes of A3A-BE3, truncated A3AΔ-BE3 variants and the recently optimized editor eA3A-BE3 (20) when targeting specific sites in the yeast Can1 gene. a Sequences of the five target sites (containing Cs at different positions). Target Cs are indicated in grey (in the middle of the sequences) and numbered relative to the PAM (grey, at the end of the sequences). Edited clones were identified by using the canavanine selection strategy (see Methods). b Base editing efficiency and precision. The x-axis represents the target Cs within the protospacers (with the order of the bars from left to right corresponding to the Cs in the legend from top to bottom). The y-axis shows their C-to-T mutation frequency (see Methods). Values and error bars represent the mean and standard deviation of three independent biological replicates.

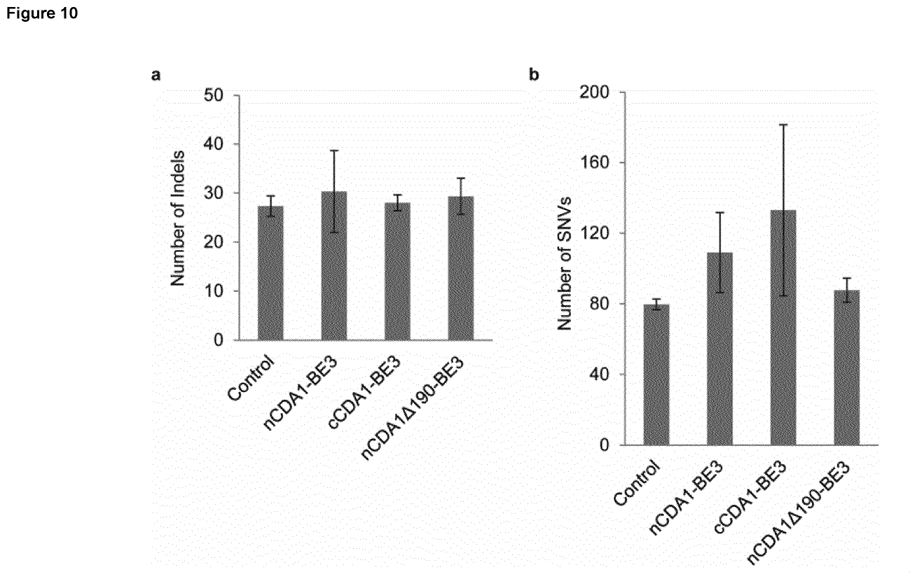

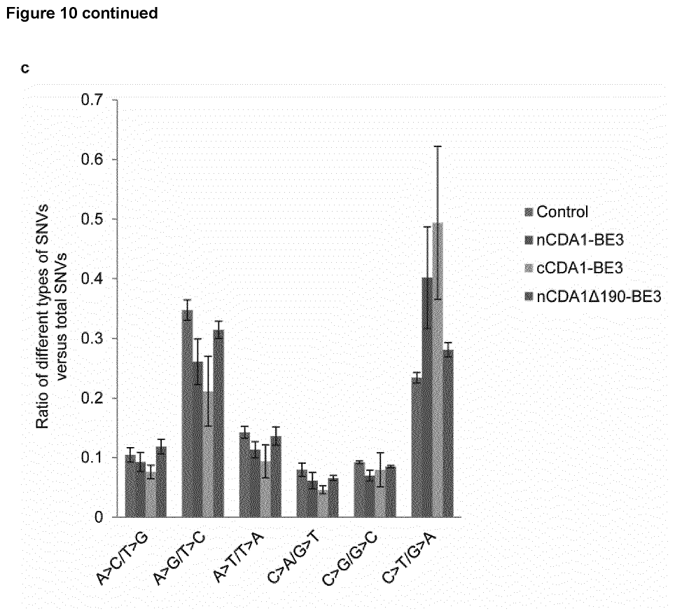

FIG. 10. Analysis of off-target editing. Genetic changes that occurred in strains harboring nCDA1-BE3, cCDA1-BE3, nCDA1Δ190-BE3 or a control plasmid without a BE construct were identified by whole genome sequencing. a-b Comparison of the total number of detected indels (a) and SNVs (b). c The mutation frequency of different types of SNVs in cells treated by the three base editors and the control. The order of the bars from left to right corresponds to the BEs listed in the legend from top to bottom. The sgRNA was designed to target site Can1-4. Values and error bars represent the mean and standard deviation of three independent biological replicates.

The examples illustrate the invention.

### Example 1

### Methods

Yeast Strains and Growth Conditions.

Saccharomyces cerevisiae BY4743 (diploid, MAT a/α, his3Δ1/his3Δ1, leu2Δ0/leu2Δ0, LYS2/lys2Δ0, met15Δ0/MET15, ura3Δ0/ura3Δ) was used as host strain for genome editing. Cells were grown non-selectively in YPAD medium (2% Bacto peptone, 1% Bacto yeast extract, 2% glucose, 0.003% adenine hemisulfate). For culture in Petri dishes, the medium was solidified with 2% agar. Selection of yeast transformants based on the URA3 and LEU2 markers was done on a synthetic complete (SC) medium (6.7 g/L of Difco Yeast Nitrogen Base, 20 g/L glucose) and a mixture of appropriate amino acids deficient in uracil and leucine (SC-U-L). Yeast strains were cultivated at 28° C. on a rotary shaker.

DNA Methods.

PCR was performed with Phusion High-Fidelity DNA Polymerase (ThermoFisher) according to the manufacturer's instructions. Cloning and amplification of plasmids were carried out in the E. coli strain DH5α. Plasmids harboring the Streptococcus pyogenes cas9 gene (p415-GaIL-Cas9-CYC1t) and a chimeric guide RNA construct (p426-SNR52p-gRNA.CAN1.Y-SUP4t) were provided by the laboratory of Dr. George Church and obtained from Addgene (Cambridge, Mass., USA).

To generate APOBECI base editors, the APOBECI reading frame and the partial cas9 sequence were PCR-amplified using oligonucleotides with overlapping linker sequences. The two fragments were cloned into the Spel/Sbfl-digested p415-GaIL-Cas9-CYC1t with the help of the In-Fusion HD Cloning Kit (Clontech, CA, USA). The D10A point mutation was introduced into cas9 with primers harboring the desired mutation by amplification of the entire plasmid template followed by DpnI digestion to remove the parental template. The UGI gene was codon-optimized for yeast and synthesized (Eurofins Genomics, Ebersberg, Germany), followed by insertion into the AscI/MluI-digested vector p415-GaIL-Cas9-CYC1t. To generate CDA1 base editors, the reading frame encoding pmCDA1 was PCR-amplified to replace the APOBECI fragment within BE3, thus generating nCDA1-BE3. To produce a fusion of CDA1 to the C-terminus of Cas9, plasmid pRS315e_pGal-nCas9 (D10A)-PmCDA1 (provided by the laboratory of Akihiko Kondo, Hyogo, Japan, and obtained from Addgene) was modified. First, the amplified UGI sequence was introduced into the XbaI site, and the resulting vector was then digested with Ascl and Sphl. Subsequently, two PCR fragments (overlapping by the XTEN linker sequence) were inserted to generate cCDA1-BE3. Insertion of three PCR fragments (covering XTEN and APOBEC1) produced base editor cBE3. The CDA1 protein truncations were generated by PCR amplification, and cloned into SpeI/Sbf1-digested BE3 or AscI/SphI-digested cBE3 vectors to produce the ΔCDA1-Cas and Cas-ΔCDA1 vector series, respectively. To produce YEE-BE3, the mutated APOBECI from plasmid pCMV-dCpf1-BE-YEE (provided by the laboratory of Jia Chen, Shanghai, China, and obtained from Addgene) was PCR amplified and cloned into SpeI/Sbf1-digested BE3.

To generate CDA1-BE3 variants with VQR-Cas9, the three required point mutations (D1135V/R1335Q/T1337R) were introduced into the cas9 gene by PCR with primers harboring the desired mutations, and the resulting three PCR products were cloned into the NruI/NcoI-digested BE3 to obtain VQR-BE3 with the help of the In-Fusion HD Cloning Kit (Clontech, Mountain View, Calif., USA). The mutated fragment was then released by digesting VQR-BE3 with NruI and MluI, followed by ligation into the similarly digested CDA1 BE plasmid (21). To construct VRER-BE3 variants, three fragments containing the four mutations (D1135V/G1218R/R1335E/T1337R) were PCR-amplified followed by cloning into the NruI/MluI-digested VQR-BE3. The mutated fragment was then excised by digesting VRER-BE3 with NruI and MluI, and ligated into the CDA1 BE construct cut with the same enzyme combination. For the generation of SpCas9-NG BE3 variants, four fragments containing the seven mutations (R1335V/L1111R/D1135V/G1218R/E1219F/A1322R/T1337R) were PCR-amplified followed by cloning into the Nrul/Mlul-digested vector VQR-BE3. The mutated fragment was released by digesting SpCas9-NG-BE3 with NruI and MluI and cloned into the similarly cut CDA1 BE plasmid. For the construction of xCas9 variants, plasmid xCas9 (3.7)-BE3 (obtained from Addgene) was digested with the restriction enzymes Sbf1 and AscI. The resulting 3.7 kb fragment was then inserted into the CDA1 BE construct digested with Sbf1 and AscI. To obtain cCDA1-BE3 variants, the mutated fragments were PCR-amplified using the corresponding BE3 variant as template and cloned into the NurI/SphI-digested cCDA1-BE3 plasmid (21).

To generate hA3A, hA3B, hA3G, hAID, mAID, cAICDA and truncated hA3A base editors, the deaminase genes were PCR-amplified from plasmid clones (provided by the laboratory of Dr. Jia Chen, Shanghai, China, and obtained from Addgene) together with part of the cas9 sequence, and then ligated into the SpeI/SbfI-digested BE3 vector. To produce A3A(R128A)-BE3, A3A(Y130F)-BE3 as well as eA3A-BE3, the point mutations (R128A, Y130F and N57G) were introduced into A3A with primers containing the appropriate mutations.

To generate plasmids expressing sgRNAs that target-specific sites, the protospacer sequences were introduced by PCR amplification, and the resulting PCR products were cloned into the Clal/Kpnl-digested vector p426-SNR52p-gRNA.CAN1.Y-SUP4t with the In-Fusion HD Cloning Kit (Clontech, CA, USA).

Yeast Transformation and Genomic DNA Extraction.

Yeast cells were transformed with the LiAc/SS carrier DNA/PEG method using 0.5-1 μg plasmid DNA (Gietz, R. D. & Schiestl, R. H. Quick and easy yeast transformation using the LiAc/SS carrier DNA/PEG method. Nat. Protoc. 2, 35-37 (2007)). Transgenic clones were selected on SC-U-L media and confirmed by PCR analyses. Yeast genomic DNA was extracted according to a published protocol (Lõoke, M., Kristjuhan, K. & Kristjuhan, A. Extraction of genomic DNA from yeasts for PCR-based applications. Biotechniques 50, 325-328 (2011)). PCR products were purified (PCR Purification kit; Macherey-Nagel) and then sequenced.

CAN1 Mutagenesis.

Yeast colonies were picked, suspended in 3 mL SC medium with 2% glucose and without leucine and uracil, and grown to a stationary phase. The cells were then pelleted, washed twice in sterile water, and then resuspended in SC induction medium with 2% galactose and 1% raffinose, but without leucine and uracil, to an OD600 of 0.3. The cells were incubated for 20 h prior to plating on YPAD rich or SC media plates without arginine but with 60 mg/mL L-canavanine (Sigma). After incubation for 3 days, the colony number on each plate was counted. The C-to-T mutation frequency in CAN1 was determined as the ratio of the colony count on canavanine-containing plates to the colony count on YPAD-rich media plates. Each experiment was performed at least three times on different days. To determine the mutation spectrum, colonies were randomly picked and suspended in sterile water, followed by PCR amplification of the relevant CAN1 fragment and DNA sequencing. Control cultures (not treated with base editors) did not produce canavanine-resistant colonies.

Next-Generation Sequencing.

Yeast colonies harboring plasmids expressing base editors and sgRNAs were picked from SC-L-U plates, suspended in 3 mL SCL-U medium with 2% glucose, and grown to a stationary phase. The cultures were then washed twice to remove residual glucose, resuspended in 5 mL SC-L-U medium with 2% galactose and 1% raffinose to an OD600 of 0.3, and incubated for 20 h at 28° C. on a rotary shaker. Genomic DNA was extracted from culture samples of 0.5 mL volume, and the regions targeted by base editing were amplified by PCR with primer pairs containing index tags for sample multiplexing. PCR amplification was performed with the Phusion High-Fidelity DNA Polymerase (ThermoFisher) according to the manufacturer's protocol, followed by product purification with the NucleoSpin Gel and PCR Clean-up kit (Machery-Nagel). The purified index-labeled PCR products were pooled at equal molar ratios. PCR-free library construction and NGS sequencing, demultiplexing by assigning reads to samples, and data filtering (including removal of adaptor sequences, contaminations and low-quality reads from raw reads) were done commercially (BGI, Hong Kong). Sequencing was performed on an Illumina MiSeq 4000 platform in a paired-end way to obtain 150 bp read length for each side and, on average, more than 100,000 reads per sample.

Data Analysis.

The clean FASTQ files obtained after data filtering were further analyzed with python scripts (available at https://github.com/zfcarpe/Cas9Sequencing). Briefly, the “pattern_extract.py” was first applied to scan all sequencing reads and extract the reads with the fixed length of the editing region (and exactly matching the two flanking sequences). This procedure excluded indel-containing and imperfectly matching reads, and allows summarizing each base calling in an alignment-like manner. Subsequent application of the “result_stat.py” script scanned each base within the editing region and calculated the frequency of each base converted to one of the other three bases by dividing the respective read number by the total number of sequencing reads to obtain the percentage of C-to-T editing and the percentage of edited reads with the C converted to any of the other bases. In addition, the script calculates the frequencies of all edited products by scanning each aligned read for conversion of the potential target cytidines. For the analysis of indel frequencies, the sequencing reads were scanned for two exactly matching 10-bp sequences that flank both sides of the region of interest (i.e., the sequence containing the editing sites). Reads without exact matches were excluded from further analysis. By calculating the length of the region, all sequencing reads exactly matching the length of the reference sequence were classified as not containing an indel, otherwise the read was classified as harboring an indel. A shell script “Cas9Sequencing.sh” combined the processes.

### Example 2

### Results

Rigid Linkers Improve Precision of APOBEC1-Based Editors.

We hypothesized that the positioning on the target sequence of the Cas9 protein relative to the deaminase domain (i.e., their physical distance) and the rigidity of the connection between these two domains of the base editor determine the width of the editing window, and hence the precision of the base editor. In previous studies, a 16 amino acid (aa) flexible linker (XTEN) has been identified as the best compromise between editing efficiency and specificity (Komor, A. C., Kim, Y. B., Packer, M. S., Zuris, J. A. & Liu, D. R. Programmable editing of a target base in genomic DNA without doublestranded DNA cleavage. Nature 533, 420-424 (2016)). Using L-canavanine selection in yeast (Nishida, K. et al. Targeted nucleotide editing using hybrid prokaryotic and vertebrate adaptive immune systems. Science 353, 1248 (2016)), we first investigated the effects of length and rigidity of the linker between APOBEC1 and nCas9 (Cas9 nickase) on base editing precision and efficiency when targeting several sites in the Can1 gene (FIG. 1) that contain Cs within the activity window of the base editor BE3 (Komor, A. C., Kim, Y. B., Packer, M. S., Zuris, J. A. & Liu, D. R. Programmable editing of a target base in genomic DNA without doublestranded DNA cleavage. Nature 533, 420-424 (2016)). L-Canavanine is a highly toxic analog of the proteinogenic amino acid arginine, and mutations inactivating the uptake protein Can1 confer resistance to canavanine. We used an inducible base editor construct, determined the optimal induction time, and then tested 10 different rigid linker sequences (containing the amino acid proline that, due to its secondary amine, confers conformational rigidity) in comparison to the commonly used XTEN flexible linker. Consistent with previous reports (Komor, A. C., Kim, Y. B., Packer, M. S., Zuris, J. A. & Liu, D. R. Programmable editing of a target base in genomic DNA without doublestranded DNA cleavage. Nature 533, 420-424 (2016)), the base editor BE3 (containing the XTEN linker) allowed editing at all Cs within a window of nine nucleotides (FIG. 1). Omission of the linker sequence or use of a very short rigid linker (i.e., the 3 aa linker PAP) abolished editing nearly completely. Interestingly, rigid linkers of 5-7 aa made editing substantially more precise, with the seven aa linker PAPAPAP largely restricting editing to positions −15 and −16 (FIG. 1). Longer linkers resulted in reduced editing accuracy, suggesting that a seven aa rigid linker is optimal.

It was reported that mutations in the APOBECI domain of BE3 can also narrow the base editing width. We, therefore, compared the base editing outcome of BE3, YEE-BE3 (the optimal BE3 variant (Kim, Y. B. et al. Increasing the genome-targeting scope and precision of base editing with engineered Cas9-cytidine deaminase fusions. Nat. Biotechnol. 35, 371-376 (2017)), and BE-PAPAPAP when targeting the Can1 sites. We found that YEE-BE3, although mainly editing C₋₁₅ or C₋₁₆, suffered from strongly reduced editing activity at these sites. Although it will be important to confirm this deficit for additional sequence contexts, this finding is consistent with a recent study that also reported low editing efficiency of the YEE-BE3 base editor (Gehrke, J. M. et al. An APOBEC3A-Cas9 base editor with minimized bystander and off-target activities. Nat. Biotechnol. 36, 977-982 (2018)).

Previous work has mostly investigated the activity of base editors in favorable sequence contexts, with relatively few C targets within the protospacer sequence. To develop a more rigorous (and Can1-independent) assay for base editor specificity, we also investigated the worst-case scenario, in which all nucleotides within the BE3 activity window are Cs (i.e., a nonacytidine motif from −13 to −21). Analysis of editing products by deep sequencing revealed that base editors with 5-7 aa rigid linkers mainly edited at positions C₋₁₄ to C₋₁₆.

These editors showed greatly improved site selectivity and a narrowed editing window, while retaining up to 90% of the editing efficiency of the original BE3.

Importantly, when editing product distribution was analyzed, BE3-treated sequences mostly contained four simultaneously edited bases, whereas short rigid linker-containing base editors predominantly generate products with one to three edited bases, thus providing further evidence for short rigid linkers leading to more precise editing.

Engineering of Improved CDA1-Based Editors.

To test whether other base editors can also be improved by engineering the linker region connecting the nucleoside deaminase domain with the nCas9 domain, we next applied a similar strategy to CDA1, the AID homolog of sea lamprey (Nishida, K. et al. Targeted nucleotide editing using hybrid prokaryotic and vertebrate adaptive immune systems. Science 353, 1248 (2016)) that has been reported to exhibit superior performance to APOBEC1 in certain sequence contexts (Komor, A. C. et al. Improved base excision repair inhibition and bacteriophage Mu Gam protein yields C:G-to-T:A base editors with higher efficiency and product purity. Sci. Adv. 3, eaao4774 (2017)).

When fused to nCas9 with flexible linkers up to 100 aa long (Nishida, K. et al. Targeted nucleotide editing using hybrid prokaryotic and vertebrate adaptive immune systems. Science 353, 1248 (2016)), CDA1 conducts C-to-T conversion in a window of approximately −16 to −19. To better understand what influences the width of the activity window, we generated four constructs for direct comparison of N- and C-terminal fusions of APOBEC1 and CDA1 to nCas9, initially using the XTEN linker (FIG. 2a). When the APOBECI domain was fused to the C-terminus of nCas9 (cBE3), the editing activity was very low (FIG. 2b, c), consistent with previous observations (Komor, A. C., Kim, Y. B., Packer, M. S., Zuris, J. A. & Liu, D. R. Programmable editing of a target base in genomic DNA without doublestranded DNA cleavage. Nature 533, 420-424 (2016)). By contrast, when CDA1 was fused to either the N-terminus or the C-terminus of nCas9, both fusions exhibited high editing efficiency. However, there was a remarkable difference in the width of the editing window, in that the N-terminal CDA1 (nCDA1-BE3) triggered editing in a much broader window when tested on either an oligo(C) substrate or target sites in the Can1 gene (FIG. 2b, c). The C-terminal fusion showed a more specific editing activity, peaking from C₋₁₆ to C₋₁₉, consistent with previous reports (Nishida, K. et al. Targeted nucleotide editing using hybrid prokaryotic and vertebrate adaptive immune systems. Science 353, 1248 (2016)).

Comparative assessment of the specificity of previously generated base editors and our base editors on several genomic target sequences showed that, in many cases, some level of discrimination between adjacent Cs is possible, but the achievable precision depends on the sequence context and on the base editor used. In general, the nCDA1-BE3 and cCDA1-BE3 editors display less dependence on the neighboring nucleotides and can edit target Cs efficiently even when located immediately after an A, a context that is only very inefficiently edited by APOBEC1-based editors. Moreover, CDA1-based editors enhance product purity, as reported previously (Komor, A. C. et al. Improved base excision repair inhibition and bacteriophage Mu Gam protein yields C:G-to-T:A base editors with higher efficiency and product purity. Sci. Adv. 3, eaao4774 (2017)).

In an attempt to further narrow the activity window of CDA1 editors, we removed the linker between CDA1 and Cas9, generating versions nCDA1-NL-BE3 and cCDA1-NL-BE3. Surprisingly, both linkerless fusions showed an unaltered activity window with largely unchanged editing efficiency at each C within it. This result suggests that the termini of CDA1 are inherently flexible and may act as linker-like sequences. We, therefore, tested the impact of N- and C-terminal truncations (removing potential linker-like fragments) on base editing.

A nuclear export signal (NES) was reported to reside in the C-terminus of the CDA1 homolog AID (Patenaude, A. M. et al. Active nuclear import and cytoplasmic retention of activation-induced deaminase. Nat. Struct. Mol. Biol. 16, 517-527 (2009)), and its location corresponds to residues 199 to 208 in CDA1 (FIG. 3a). Deletion of the NES from AID increased the deamination efficiency of the enzyme (Yang, L. et al. Engineering and optimising deaminase fusions for genome editing. Nat. Commun. 7, 1038 (2016); Ma, Y. et al. Targeted AID-mediated mutagenesis (TAM) enables efficient genomic diversification in mammalian cells. Nat. Methods 13, 1029-1035 (2016); Hess, G. T. et al. Directed evolution using dCas9-targeted somatic hypermutation in mammalian cells. Nat. Methods 13, 1036-1042 (2016)). We generated a series of 22 base editors with C-terminally truncated CDA1 versions fused to nCas9 (FIG. 3b) and tested them on two oligo(C) motifs (FIG. 4). While removal of the NES had only small effects on editing efficiency and specificity (nCDA1Δ198-BE3), larger deletions made editing more precise and substantially narrowed the activity window of the base editors (FIG. 4). The enzyme tolerated truncations up to amino acid residue 158 without a significant loss in editing efficiency (FIG. 4). The major gain in site selectivity was seen with the removal of at least 13-14 amino acids from the Cterminus of CDA1 (nCDA1Δ195-BE3, nCDA1Δ194-BE3; FIG. 4). Larger deletions had similar beneficial effects on editing precision, although some of them displayed slightly reduced overall editing efficiency (FIG. 4). Unlike the full-length base editor, the best-performing truncated variants showed a clear preference for one or two Cs within the oligo(C) stretch (e.g., nCDA1Δ194-BE3 for C₋₁₈ and, to a lesser extent, C₋₁₇ within the (C)₉ motif: FIG. 4a; nCDA1Δ192-BE3 and nCDA1Δ190-BE3 for C₋₁₈ in the (C)₈ motif: FIG. 4b). By contrast, truncations at the N-terminus of CDA1 in cCDA1-BE3 had no significant effect on the width of the editing window.

Tests on oligo(C) motifs represent the most stringent assays for site selectivity of base editors. However, such long C stretches would only rarely be targets of genome editing with base editors in vivo. To assess whether base editors with C-terminally truncated CDA1 domains also show superior performance in more natural (heteropolymeric) genomic sequence contexts, we targeted four sites in the Can1 gene, each of which contains at least one additional C directly adjacent or close to position C₋₁₈. When the base editing outcome of nCDA1-BE3, cCDA1-BE3 and our base editors with truncated CDA1 domains were compared, our base editors displayed editing with much higher precision (FIG. 5). For all four tested sites, our base editors mainly edited position C₋₁₈, with a 2- to 20-fold higher efficiency than other adjacent Cs (FIG. 5a). Importantly, the base editors also produced predominantly single-C-modified products at position C₋₁₈ (accounting for 50-94% of all edited products), whereas nCDA1-BE3 and cCDA1-BE3 produced mainly double or triple modified products (FIG. 5b, c). We also investigated the indel frequency and base editing purity at these sites when treated by narrowed-window base editors. We found that the frequency of editing errors was very low, consistent with what has been reported for other base editors.

Finally, we also determined the base editing outcome in individual colonies obtained by the canavanine selection method. While nCDA1-BE3 and cCDA1-BE3 yielded only 1 and 6 colonies (out of total 24 randomly picked colonies), respectively, that carried the specifically C₋₁₈ edited Can1 gene biallelically (i.e., in a homozygous fashion), the base editors with truncated CDA1 domains yielded 18-24 colonies that were homozygous for the allele only edited at position C₋₁₈. Importantly, two of the base editors produced 100% precisely edited homozygous clones (FIG. 6; Table 1).

TABLE 1
Base editors with CDA1 truncations exhibit many more homozygous C₋₁₉T₋₁₈
colonies than nCDA1-BE3 and cCDA1-BE3*. For each base editor, 24 canavanine-resistant
colonies were randomly picked from the selection plate followed by sequencing of the Can1
locus. The major types of edited products are listed in the first column of the table, and the
colony numbers representing each product type are given. For nCDA1-BE3, the genotype of
the remaining colony is C₋₁₉T₋₁₈/T₋₁₉C₋₁₈; for nCDA1Δ194-BE3, the remaining two colonies are
C₋₁₉T₋₁₈/T₋₁₉C₋₁₈ and T₋₁₉T₋₁₈/T₋₁₉C₋₁₈, respectively.

| | nCDA1- | cCDA1- | nCDA1Δ194- | nCDA1Δ193- | nCDA1Δ192- | nCDA1Δ190- | nCDA1Δ184- | nCDA1Δ176- |
|---|---|---|---|---|---|---|---|---|
| | BE3 | BE3 | BE3 | BE3 | BE3 | BE3 | BE3 | BE3 |
| C₋₁₉T₋₁₈ | 1/24 | 6/24 | 18/24 | 21/24 | 22/24 | 24/24 | 24/24 | 20/24 |
| Homozygous | | | | | | | | |
| C₋₁₉T₋₁₈/T₋₁₉T₋₁₈ | 0/24 | 11/24 | 2/24 | 2/24 | 1/24 | 0/24 | 0/24 | 2/24 |
| Heterozygous | | | | | | | | |
| T₋₁₉C₋₁₈ | 22/24 | 7/24 | 2/24 | 1/24 | 1/24 | 0/24 | 0/24 | 2/24 |
| Homozygous | | | | | | | | |

Expanding Precision Base Editing to Non-NGG PAM Sequences

Recently, several Cas9 variants have been described that recognize non-NGG PAM sequences (Nishimasu, H. et al. Engineered CRISPR-Cas9 nuclease with expanded targeting space. Science 361, 1259-1262 (2018); Hu, J. H. et al. EvolvedCas9 variants with broad PAM compatibility and high DNA specificity. Nature 556, 57-63 (2018); Kleinstiver, B. P. et al. Engineered CRISPR-Cas9 nucleases with altered PAM specificities. Nature 523, 481-485 (2015)). To test whether Cas9 variants with expanded PAM compatibility can be used in our high-precision BEs to extend their DNA targeting scope, we replaced the nCas9 sequence with that of four different nCas9 variants recognizing four different non-NGG PAMs (FIG. 7a, b). Of particular interest is the minimal PAM sequence NG (as recognized by variant SpCas9-NG; FIG. 7b), which occurs much more frequently in DNA sequences than the wild-type PAM sequence NGG. As deaminase domain, we tested the full-length CDA1 and a series of truncated CDA1 versions that lack 13 to 20 C-terminal amino acids. When fused to nCas9, this range of C-terminal deletions was shown previously to provide the maximum increase in editing precision while retaining high editing activity (21). In this way, 32 new BEs were constructed: the full-length CDA1 (as N-terminal or C-terminal fusion) and 6 CDA1 deletions combined with the VQR-Cas9 variant (nCDA1Δ195-VQRBE3; nCDA1Δ194-VQRBE3; nCDA1Δ193-VQRBE3; nCDA1Δ192-VQRBE3; nCDA1Δ190-VQRBE3; nCDA1Δ188-VQRBE3; FIG. 7a, c) that recognizes the PAM sequence NGA (FIG. 7b), the full-length CDA1 (as N-terminal or C-terminal fusion) and 6 CDA1 deletions combined with the VRER-Cas9 variant (nCDA1Δ195-VRERBE3; nCDA1Δ194-VRERBE3; nCDA1Δ193-VRERBE3; nCDA1Δ192-VRERBE3; nCDA1Δ190-VRERBE3; nCDA1Δ188-VRERBE3; FIG. 7d) that recognizes the PAM sequence NGCG (FIG. 7b), the full-length CDA1 (as N-terminal or C-terminal fusion) and 6 CDA1 deletions combined with the xCas9 variant (nCDA1Δ195-xBE3; nCDA1Δ194-xBE3; nCDA1Δ193-xBE3; nCDA1Δ192-xBE3; nCDA1Δ190-xBE3; nCDA1Δ188-xBE3; FIG. 7e) that recognizes the PAM sequences NG, GAA and GAT (FIG. 7b), and the full-length CDA1 (as N-terminal or C-terminal fusion) and 6 CDA1 deletions combined with the SpCas9-NG variant (nCDA1Δ195-NGBE3; nCDA1Δ194-NGBE3; nCDA1Δ193-NGBE3; nCDA1Δ192-NGBE3; nCDA1Δ190-NGBE3; nCDA1Δ188-NGBE3; FIG. 7f,g) that recognizes the PAM sequence NG (FIG. 7b).

For each set of BEs, we tested target sites that contain a stretch of consecutive cytidines within the activity window upstream of the PAM. PolyC motifs were used to provide the most rigorous test for editing precision, in that specific editing of a single C would require maximum discriminatory power. Editing efficiency and precision were first assessed by dideoxy chain termination sequencing of amplified PCR products, and the two best-performing BEs were then further characterized by high-throughput next-generation sequencing (FIG. 7; see Methods; Tan, J., et al. Engineering of high-precision base editors for site-specific single nucleotide replacement. Nat. Commun. 10, 439 (2019)).

The VQR-Cas9 variant recognizes the PAM sequence NGA (FIG. 7b). The activity window ranged from C₋₁₄ to C₋₁₉ in target sequence PolyC-1-NGA and from C₋₁₄ to C₋₂₀ in target sequence PolyC-2-NGA. By contrast, VQR-Cas9 BEs harboring CDA1 truncations had a much narrower activity window and predominantly edited positions C₋₁₇ and C₋₁₈ in target sequence PolyC-1-NGA and C₋₁₇ and C₋₁₈ in sequence PolyC-2-NGA (FIG. 7c). Interestingly, the largest truncation, nCDA1Δ188-VQRBE3, even discriminated to some extent between the two positions in that C₋₁₈ was edited nearly twice as efficiently as C₋₁₇ in sequence PolyC-1-NGA (FIG. 7c).

The VRER-Cas9 variant recognizes the PAM sequence NGCG (FIG. 7b). The truncated variants efficiently edited both target sequences and displayed greatly superior editing precision on sequence PolyC-4-NGCG (FIG. 7d).

Recently, two Cas9 variants, designated xCas9 and SpCas9-NG, were developed that show greatly relaxed PAM recognition specificity and, instead of NGG, recognize the minimal PAM sequence NG (Nishimasu, H. et al. Engineered CRISPR-Cas9 nuclease with expanded targeting space. Science 361, 1259-1262 (2018); Hu, J. H. et al. EvolvedCas9 variants with broad PAM compatibility and high DNA specificity. Nature 556, 57-63 (2018)). When tested on three non-NGG target sites (PolyC-1-NGA, PolyC-5-NGC and PolyC-6-NGT), xCas9-derived BEs displayed detectable activity only on one of the three sites (PolyC-5-NGC; FIG. 7e. A particularly well-performing truncated variant, nCDA1Δ194-xBE3, edited position C₋₁₈ with high selectivity and strongly enhanced efficiency (of more than 35%; FIG. 7e).

BEs constructed with SpCas9-NG edited all three non-NGG target sites (FIG. 7f,g). Compared to the full-length BE (nCDA1-NGBE3), the truncated versions again exhibited superior editing preference. The truncated versions predominantly edited one or two nucleotides (FIG. 7f,g). Typically, position C₁₈ was most efficiently recognized, but dependent on the target site, some BEs also edited C₋₁₇ (e.g., nCDA1Δ194-NGBE3 in PolyC-1-NGA) or C₋₁₉ (e.g., nCDA1Δ194-NGBE3 in PolyC-6-NGT; FIG. 7g) at high efficiency. For comparison, we also tested the reciprocal fusions harboring the SpCas9 variants at the N-terminus (cCDA1-VQRBE3, cCDA1-VRERBE3, cCDA1-xBE3 and cCDA1-NGBE3). These fusions showed a narrower activity window than the C-terminal fusions, but did not reach the specificity of the best-performing fusions with truncated CDA1 versions. When target sites upstream of the wild-type PAM of Cas9, NGG, were tested, the SpCas9-NG-derived BEs displayed reduced editing activity compared to wild-type Cas9-derived BEs. This finding is consistent with recent studies that reported lower genome editing activity of SpCas9-NG on canonical NGG PAMs (Nishimasu, H. et al. Engineered CRISPR-Cas9 nuclease with expanded targeting space. Science 361, 1259-1262 (2018), Zhong, Z. et al. Improving plant genome editing with high-fidelity xCas9 and non-canonical PAM-targeting Cas9-NG. Mol. Plant 12, 1027-1036 (2019)).

Taken together, our findings indicate that BEs with truncated CDA1 sequences tolerate replacement of Cas9 with variants that recognize alternative PAMs, including PAMs with greatly relaxed specificity such as NG. The high efficiency and accuracy of these new editors greatly expand the editing scope of high-precision BEs.

Engineering of A3A-Based Precision BEs

In an attempt to develop additional high-precision BEs that selectively edit nucleotide positions other than C₋₁₈, we generated fusions of several deaminases to nCas9 by omitting a linker sequence between the two proteins. This approach was taken to investigate the possibility that these deaminases inherently harbor a linker-like fragment at their C-terminus.

Six different deaminases were tested by fusing nCas9 directly to their C-terminus. The fusion proteins were then assayed for their base editing efficiency on two polyC-containing target sites. The BE based on the human cytidine deaminase APOBEC3A (A3A; (Gehrke, J. M. et al. An APOBEC3A-Cas9 base editor with minimized bystander and off-target activities. Nat. Biotechnol. 36, 977-982 (2018)), referred to as hA3A-NL-BE3, displayed the best performance in that it conferred the highest editing efficiency on both target sequences. We, therefore chose A3A for further optimization.

For comparison, we also generated an A3A-BE3 editor with the standard XTEN linker (Rees, H. A. et al. Improving the DNA specificity and applicability of base editing through protein engineering and protein delivery. Nat. Commun. 8, 15790 (2017)). Surprisingly, we observed that hA3A-NL-BE3 (for brevity subsequently referred to as A3A-NL-BE3) showed a slightly broader editing window than A3A-BE3 and also caused a shift in the most strongly edited (central) positions, despite the shorter connection between the cytidine deaminase domain (A3A) and the nCas9 domain of the fusion protein. This may be attributable to linker removal slightly altering the spatial structure of the fusion protein (and, in this way, affecting positioning of the deaminase domain on the target sequence), and would be consistent with the variable effects of linker engineering seen in previous studies (Kim, Y. B., et al. Increasing the genome-targeting scope and precision of base editing with engineered Cas9-cytidine deaminase fusions. Nat. Biotechnol. 36, 371-376 (2017); Tan, J., et al. Engineering of high-precision base editors for site-specific single nucleotide replacement. Nat. Commun. 10, 439 (2019)). The editing efficiency of both BEs was similar at both tested sites (Supplementary FIG. 10), possibly suggesting that the C-terminus of A3A is extraordinarily flexible.

A3A-based BEs were reported to exhibit a lower dependence on the sequence context, reduced sensitivity to DNA methylation and a wider editing window (Zong, Y. et al. Efficient C-to-T base editing in plants using a fusion of nCas9 and human APOBEC3A. Nat. Biotechnol. 36, 950-953 (2018); Wang, X., et al. Efficient base editing in methylated regions with a human APOBEC3A-Cas9 fusion. Nat. Biotechnol. 36, 946-949 (2018); Gehrke, J. M. et al. An APOBEC3A-Cas9 base editor with minimized bystander and off-target activities. Nat. Biotechnol. 36, 977-982 (2018)). To test if the precision of these BEs can be improved by narrowing the activity window, we constructed a series of truncations at the C-terminus of A3A and determined their impact on base editing (FIG. 8a). Previously, we showed that the major gain in site selectivity for CDA1-based BEs was seen with the removal of at least 13 amino acids from the C-terminus (nCDA1Δ195-BE3; Tan, J., et al. Engineering of high-precision base editors for site-specific single nucleotide replacement. Nat. Commun. 10, 439 (2019)). Alignment of A3A with CDA1 revealed that the 13 amino acid CDA1 truncation corresponds to residue 194 of A3A. We generated six BEs with C-terminally truncated A3A versions fused to nCas9 and tested them on two polycytidine motifs (FIG. 8b). Deletion of 17 amino acids (A3AΔ182-BE3) made the editing significantly more specific in that A3AΔ182-BE3 preferentially edits position C₋₁₅ or C₋₁₆ (FIG. 8b). When tested on target sequence polyC-8, the truncated editors A3AΔ190-BE3, A3AΔ186-BE3 and A3AΔ182-BE3 displayed improved specificity. For example, A3AΔ182-BE3 exhibits a strong preference for positions C₋₁₅ and C₋₁₆, while showing greatly reduced editing activity at the neighboring positions C₋₁₇ and C₋₁₄ (FIG. 8c).

To confirm the superior precision of the truncated editors A3AΔ190-BE3, A3AΔ186-BE3 and A3AΔ182-BE3, we compared the base editing outcomes when targeting different cytidines within the yeast Can1 gene (Tan, J., et al. Engineering of high-precision base editors for site-specific single nucleotide replacement. Nat. Commun. 10, 439 (2019)). Each of the five tested sites contains one or two target Cs in different distances from the PAM, ranging from position C₋₁₉ to position C₋₁₁ (FIG. 9a). Canavanine-resistant colonies clones can arise only when C-to-T base editing occurs and results in synthesis of an inactive gene product (Tan, J., et al. Engineering of high-precision base editors for site-specific single nucleotide replacement. Nat. Commun. 10, 439 (2019)). While the BE with the full-length A3A (A3A-BE3) non-selectively edited all Cs within a window of nine nucleotides (FIG. 9b), the BEs containing truncated A3A versions mainly edited positions C₋₁₅ or C₋₁₆, confirming the results obtained with polycytidine target sequences (FIG. 8b).

It was recently reported that mutations in A3A (N57G mutation in an A3A variant dubbed eA3A) can reduce bystander editing frequency by enhancing the preference of the editor for TCR motif (with R being A or G; Gehrke, J. M. et al. An APOBEC3A-Cas9 base editor with minimized bystander and off-target activities. Nat. Biotechnol. 36, 977-982 (2018)). We, therefore, generated an eA3A-BE3 editor and compared it with our best-performing truncated A3A BEs. We found that eA3A, although mainly editing C₋₁₅ or C₋₁₆, suffered from reduced editing activity (FIG. 9b), suggesting relatively poor editing at non-TCR sites.

It has been reported that A3A-derived BEs can induce significant transcriptome-wide off-target editing at the RNA level. Specific amino acid substitutions (R128A or Y130F) in A3A largely eliminate these off-target activities (Zhou, C., et al. Off-target RNA mutation induced by DNA base editing and its elimination by mutagenesis. Nature 571, 275-278 (2019); Grünewald, J., et al. Transcriptome-wide off-target RNA editing induced by CRISPR-guided DNA base editors. Nature 569, 433-437 (2019)). We therefore investigated the effect of each of these two mutations on the width of the base editing window and the BE activity when combined with proper A3A truncations. Introduction of either of the two mutations into A3A-BE3 neither reduced the base editing efficiency, consistent with previous findings (Zhou, C., et al. Off-target RNA mutation induced by DNA base editing and its elimination by mutagenesis. Nature 571, 275-278 (2019)), nor did it affect the base editing window. When we combined these mutations with the two optimal A3A truncations (A3AΔ186 and A3AΔ182), we found that Y130F, but not R128A, in combination with the A3A version truncated at residue 186 (i.e., BE variant A3A(Y130F)Δ186-BE3) displays a base editing window and an editing efficiency similar to A3AΔ186-BE3, and thus should be used to suppress off-target RNA editing.

Together, these data demonstrate that the A3A deaminase can be engineered to obtain high-precision base editors that predominantly edit position C₋₁₅ or C₋₁₆, while retaining high editing efficiency.

Analysis of Genome-Wide Off-Target Editing by Whole Genome Sequencing

Recently, cytosine base editors were reported to produce substantial genome-wide off-target effects that are largely independent of the sgRNA (Jin, S., et al. Cytosine, but not adenine, base editors induce genome-wide off-target mutations in rice. Science 364, 292-295 (2019); Zuo, E., et al. Cytosine base editor generates substantial off-target single-nucleotide variants in mouse embryos. Science 364, 289-292 (2019)). Since a narrower editing window means fewer target nucleotides, we envisioned that our narrow-window base editors could also reduce the off-target DNA editing. We, therefore, investigated off-target editing in yeast cells treated with nCDA1-BE3, cCDA1-BE3, nCDA1Δ190-BE3 and a no BE control, in combination with an sgRNA targeting a Can1 site. Canavanine selection was used to isolate colonies harboring on-target editing events. The truncated CDA1 version Δ190 was chosen for this experiment, because we had previously shown that this version displays high editing precision as well as high editing efficiency for most tested sites (Tan, J., et al. Engineering of high-precision base editors for site-specific single nucleotide replacement. Nat. Commun. 10, 439 (2019)). For all constructs, cultures grown from three different transformed colonies were mixed, followed by genomic DNA isolation and whole-genome sequencing. The three BE variants showed comparable numbers of indels as the no BE control (FIG. 10a). When the total number of SNVs (single nucleotide variants) was analyzed, the full-length fusions were found to display many more SNVs than the control, in agreement with the previous reports on off-target effects of cytosine BEs (Jin, S., et al. Cytosine, but note adenine, base editors induce genome-wide off-target mutations in rice. Science 364, 292-295 (2019); Zuo, E., et al. Cytosine base editor generates substantial off-target single-nucleotide variants in mouse embryos. Science 364, 289-292 (2019)). However, the truncated version exhibited a substantially reduced number of SNVs that was only slightly higher than that of the negative control (FIG. 10b). We also analyzed the mutation types and found that, in nCDA1-BE3 and cCDA1-BE3, the frequency of C-to-T (G-to-A) transitions was significantly higher than in the control and the truncated base editor nCDA1Δ190-BE3 (FIG. 10c). These findings indicate that high editing precision of BEs can contribute to reduced non-specific editing at off-target sites.

Guidelines for the Choice of the Optimal Cytidine BE

Three different cytidine deaminases (APOBEC1, CDA1 and APOBEC3A) have been engineered to produce efficient cytosine BEs, modify PAM specificities, and alter position and width of the editing window. BE variants with different properties have been obtained that differ in their suitability for (i) different target sequences and (ii) different positions of the C to be edited within the protospacer.

There is now sufficient information available to define some guidelines for the choice of the best BE depending on the position of the C, the sequence context and the presence or absence of bystander Cs (see Table 1 which is presented further above).

If the target C is located at position C₋₁₉ relative to the PAM and no bystander C is present, three BEs can be recommended: nCDA1-BE3, nCDA1Δ198-BE3 and A3A-NL-BE3. If the target C is in the same position (C₋₁₉), but has a bystander C directly upstream (CCDDD motif, with D being any nucleotide but C), cCDA1-BE3 would be the best choice (Tan, J., et al. Engineering of high-precision base editors for site-specific single nucleotide replacement. Nat. Commun. 10, 439 (2019)).

If the target C is located at C₋₁₈ and has a bystander C in its vicinity (NCN motif, with N being any nucleotide, including a possible bystander C), BEs with C-terminal truncations of CDA1 (Δ194 to Δ188) are recommended (FIG. 7; Tan, J., et al. Engineering of high-precision base editors for site-specific single nucleotide replacement. Nat. Commun. 10, 439 (2019)), and it may be advisable to test two or three different truncations.

For editing at C₋₁₆ with a 5′ bystander C (NCD context) or editing at C₋₁₅ with a 3′ bystander C (DCN), A3AΔ182-BE3 and A3A(Y130F)Δ186-BE3 are the editors of choice (FIGS. 2 and 4; Table 1).

With our set of narrow-window BEs, many disease-causing T-to-C and A-to-G mutations can now potentially be corrected in a precise manner. For example, a T-to-C mutation at position 497 of the coding region of the human gene encoding presenilin-1 (PSEN1-L166P mutation) is associated with early-onset Alzheimer's disease (Moehlmann, T., et al. Presenilin-1 mutations of leucine 166 equally affect the generation of the Notch and APP intracellular domains independent of their effect on Abeta 42 production. Proc. Natl. Acad. Sci. USA 99, 8025-8030 (2002)). This mutation can be corrected by a BE that has this C within its predicted editing window at position −18 relative to the PAM sequence NG. Precision is important here, because an additional C is present immediately adjacent to the target C (at position 496), which also lies within the editing window (−19 relative to the PAM). Using precision BEs with CDA1 truncations, this C now can be targeted much more accurately (Table 1). Similarly, an A-to-G mutation at position 980 of the coding region of the tyrosinase-encoding gene (representing a T-to-C mutation in the complementary strand) causes oculocutaneous albinism (TYR-Y327C mutation; 8). The target C is in a TCAC motif and located in position −15 of the PAM sequence AGG. Therefore, this mutation can be precisely corrected with the BEs A3AΔ182-BE3 or A3A(Y130F)Δ186-BE3 (Table 1).

1. A base editing compound comprising or consisting of
(a) a Cas protein, and, covalently connected therewith;
(b) a nucleobase-modifying enzyme,
wherein the covalent connection of (a) and (b) is
(i) direct;
(ii) provided by a peptide comprising at least one Pro residue, said peptide having a length between 1 and 20, preferably between 1 and 15 amino acids; or
(iii) provided by a non-peptidic linker, said non-peptidic linker being a small organic molecule comprising one or more double bonds, one or more triple bonds, and/or one or more aromatic rings.

2. The compound of claim 1, wherein
(a) said Cas protein is a Cas nickase or dead Cas, said Cas nickase preferably being Cas9 or Cas12, and said dead Cas preferably being dead Cas9 or dead Cas12; and/or
(b) said nucleobase-modifying enzyme is selected from a deaminase, a nucleoside synthase, a DNA methyl transferase and a DNA demethylase, said deaminase preferably being selected from the APOBEC, CDA1 or Tad/ADAR families, APOBEC3A being particularly preferred.

3. The compound of claim 1 or 2, wherein said deaminase is truncated at the N- and/or C-terminus, wherein in case of APOBEC deaminases C-terminal truncation is preferred, an APOBEC3A with a C-terminal truncation of 17 amino acids (A3AΔ182) being particularly preferred, and in case of CDA1 deaminases, truncations from residue 188 or residue 198 onwards being preferred.

4. The compound of claim 3, wherein the truncated residues are not essential for catalytic activity of said deaminase.

5. The compound of any one of claims 1 to 4, wherein said compound comprises said peptide and wherein said peptide
(i) consists of an amino acid sequence comprising 1 to 10 Pro residues and 1 to 10 small amino acid residues, said small amino acid residues preferably being selected from Ala, Gly and Ser, said amino acid sequence preferably being the sequence of SEQ ID NO: 130 (XTEN);
(ii) has a length of 5, 6 or 7 amino acids; and/or
(iii) consists of the sequence Aₘ(PA)ₙPₚ, wherein m and p are independently 0 or 1 and n is 1, 2, 3, 4, 5, 6 or 7, for example of SEQ ID NO: 154 or 162.

6. The compound of any of the preceding claims, said compound comprising or further consisting of one or both of
(a) an inhibitor of base excision repair, preferably an uracil DNA glycosylase inhibitor (UGI), more preferably the sequence of SEQ ID NO: 149; and
(b) a nuclear localization signal (NLS), preferably the sequence of SEQ ID NO: 135;
wherein (a) and/or (b) are preferably connected to each other and/or to said Cas protein with a peptidic linker consisting of 1 to 10 amino acids, said linker preferably consisting of the sequence of SEQ ID NO. 132 or 148.

7. The compound of any one of the preceding claims, wherein
(a) said deaminase is APOBEC3A (A3A; SEQ ID NO: 183), wherein preferably said A3A is truncated at the C-terminus;
(b) said deaminase is A3AΔ182 (SEQ ID NO: 205) and is fused to the N-terminus of said Cas protein;
(c) said deaminase is APOBEC1, preferably consisting of the sequence of SEQ ID NO: 129, and is fused to the N-terminus of said Cas protein; or
(d) said deaminase is CDA1, preferably consisting of the sequence of SEQ ID NO: 137; and is fused to the N-terminus or the C-terminus, preferably to the N-terminus of said Cas protein, wherein preferably the C-terminus of said CDA1 is truncated, preferably either from position 198 onwards or from any of positions 188 to 194 onwards.

8. The compound of any one of the preceding claims, wherein
(a) said Cas protein consists of the amino acid sequence of SEQ ID NO: 1 or a sequence with at least 80% identity thereto and preferably providing nickase activity, or is encoded by the nucleic acid sequence of SEQ ID NO: 2 or a sequence with at least 80% thereto identity and preferably encoding a protein with nickase activity, and is preferably selected from VQR-Cas9 (amino acid sequence of SEQ ID NO: 121), VRER-Cas9 (amino acid sequence of SEQ ID NO: 122), xCas9 (amino acid sequence of SEQ ID NO: 123), and Cas9-NG (amino acid sequence of SEQ ID NO: 124) or encoded by any one of SEQ ID NOs: 23, 24, 25 or 26; and/or
(b) said deaminase consists of the amino acid sequence of any one of SEQ ID NOs: 205, 129, 137, 169, 176, 183, 198, 212, 219, 3 and 5, a sequence with at least 80% identity and providing deaminase activity or a truncated version of any such sequence, or is encoded by the nucleic acid sequence of any one of SEQ ID NOs: 107, 31, 55, 71, 78, 85, 100, 114, 220, 4 and 6, a sequence with at least 80% identity and encoding a protein with deaminase activity or a truncated version of any such sequence.

9. The compound of any one of the preceding claims, wherein said compound is a single polypeptide and comprises or consists of an amino acid sequence selected from SEQ ID NOs: 204, 7, 9, 11, 13, 136, 218, 190, 144, 168, 182, 128, 152, 204 and 211.

10. A nucleic acid encoding the compound of any one of the preceding claims, to the extent said compound is a single polypeptide.

11. A method of base editing, said method comprising introducing into a cell a nucleic acid of claim 10 or a compound of any one of claims 1 to 9.

12. The method of claim 11, further comprising introducing into said cell a guide nucleic acid for said nickase.

13. The method of claim 11 or 12, wherein said method is performed in vitro or ex vivo.

14. A pharmaceutical composition comprising or consisting of
(a) the compound of any one of claims 1 to 9; and/or
(b) the nucleic acid of claim 10.

15. The pharmaceutical composition of claim 14, further comprising or further consisting of a guide nucleic acid for said nickase, wherein said guide nucleic acid comprises a sequence which is homologous to a subsequence of a target gene, wherein said target gene is associated with a genetic disorder.

16. A compound of any one of claims 1 to 9 or a nucleic acid of claim 10, and a guide nucleic acid for said nickase for use in a method of treating, alleviating or preventing a disorder, wherein said guide nucleic acid comprises a sequence which is homologous to a subsequence of a target gene, wherein said disorder is associated with a point mutation or an SNP in said target gene.

17. A kit comprising or consisting of
(a) (i) one or more compounds of any one of claims 1 to 9; and/or
(ii) one or more nucleic acids of claim 10.

18. The kit of claim 17, furthermore comprising or further consisting of
(b) one or more guide nucleic acids for the nickase comprised in said compound, wherein each of said guide nucleic acids comprises a sequence which is identical to a subsequence of a given target gene; and/or
(c) a manual comprising instructions for performing the method of any one of claims 11 to 13.

19. The kit of claim 17 or 18, wherein said kit comprises a plurality of said compounds and/or a plurality of said nucleic acids, wherein at least two of said compounds of (a)(i) or at least two of the compounds encoded by said nucleic acids of (a)(ii) differ with regard to their base editing profile.

20. Use of a peptide as defined in any one of the preceding claims or of a non-peptidic linker as defined in claims 1 and 9 for covalently connecting a Cas protein such as a Cas nickase (nCas) or a dead Cas (dCas) and a deaminase (DA) to provide a base editing compound.

21. The use of claim 20, wherein said deaminase is truncated at the N- or C-terminus.
